(19) United States
(12) Patent Application Publication
(10) Pub. No.: US 2026/0337171 A1
(43) Pub. Date: Oct. 29, 2026

(54) CRYSTALLINE FORMS OF {2-[3-CYCLOHEXYL-3-(TRANS-4-PROPOXY-CYCLOHEXYL)-UREIDO]-THIAZOL-5-YLSULFANYL}-ACETIC ACID AND USES THEREOF

(71) Applicant: vTv Therapeutics LLC, High Point, NC (US)

(72) Inventors: Jing TENG, Lafayette, IN (US); Yizheng CAO, Lafayette, IN (US)

(21) Appl. No.: 19/717,308

(22) Filed: Jun. 23, 2026

Related U.S. Application Data
(63) Continuation of application No. 18/000,908, filed on Jun. 7, 2021, now Pat. No. 12,709,603.
(60) Provisional application No. 63/035,994, filed on Jun. 8, 2020.

(51) Int. Cl. C07D 277/54; A61K 31/426

(52) U.S. Cl. CPC C07D 277/54; A61K 31/426; C07B 2200/13

(57) ABSTRACT

The present disclosure relates to a) crystalline forms of {2-[3-cyclohexyl-3-(trans-4-propoxy-cyclohexyl)-ureido]-thiazol-5-ylsulfanyl}-acetic acid (“Compound I”); b) pharmaceutical compositions, comprising one or more crystalline forms of Compound I, and optionally, a pharmaceutically acceptable carrier; c) methods of treating a type of diabetes mellitus or other disorders by administering one or more crystalline forms of Compound I; and d) methods for the preparation of crystalline forms of Compound I.

### CROSS REFERENCE

This application is a continuation of application Ser. No. 18/000,908, filed Dec. 6, 2022, which is a U.S. National Stage Application of International Application No. PCT/US2021/036082, filed Jun. 7, 2021, which claims the benefit of U.S. Provisional Application No. 63/035,994, filed Jun. 8, 2020, each of which are incorporated herein by reference in their entirety.

### FIELD OF THE INVENTION

The present disclosure relates to a) crystalline forms of {2-[3-cyclohexyl-3-(trans-4-propoxy-cyclohexyl)-ureido]-thiazol-5-ylsulfanyl}-acetic acid (“Compound I” or “API”); b) pharmaceutical compositions, comprising one or more crystalline forms of {2-[3-cyclohexyl-3-(trans-4-propoxy-cyclohexyl)-ureido]-thiazol-5-ylsulfanyl}-acetic acid, and optionally, a pharmaceutically acceptable carrier; and c) methods of treating a type of diabetes mellitus and other disorders by administering one or more crystalline forms of {2-[3-cyclohexyl-3-(trans-4-propoxy-cyclohexyl)-ureido]-thiazol-5-ylsulfanyl}-acetic acid to a subject in need thereof.

### BACKGROUND

Glucokinase (“GK”) is a key regulator of glucose homeostasis and acts as the physiological glucose sensor, changing its conformation, activity, and/or intracellular location in parallel with changes in glucose concentrations. GK has two main distinctive characteristics that make it a good choice for blood glucose control. First, its expression is mostly limited to tissues that require glucose-sensing (mainly liver and pancreatic β-cells). Second, GK is able to sense changes in serum glucose levels and modulate changes in liver glucose metabolism that in turn regulate the balance between hepatic glucose production (HGP) and glucose consumption, and to modulate changes in insulin secretion by the beta-cells. The concept of GK activation for the treatment of diabetes is attractive because it has proven to be effective and safe in normalizing glycemia in animal models of type 1 and type 2 diabetes by a mechanism entirely distinct from the action of antidiabetic therapies currently on the market.

Although multiple small-molecule activators of GK have been in clinical development, their initial therapeutic promise has been hampered by the occurrence of hypoglycemia, increased triglycerides (TG) concentrations, and loss of efficacy over time. These adverse events (AEs) were related to ongoing β cell activation. Compound I, a hepatoselective agent, does not cause similar adversarial effects. (Vella et al., Science Translational Medicine 16 Jan. 2019).

Compound I is an oral, small molecule, liver selective glucokinase activator that improves glycemic control and may not induce hypoglycemia, dyslipidemia, or pathological increases of glycogen and TG in the liver at therapeutically relevant doses. (Vella et al., Science Translational Medicine 16 Jan. 2019).

Not all compounds that are GK activators have characteristics affording the best potential to become useful therapeutics. Some of these characteristics include high affinity at the glucokinase, duration of glucokinase activation, oral bioavailability, tissue distribution, and stability (e.g., ability to formulate or crystallize, shelf life). Favorable characteristics can lead to improved safety, tolerability, efficacy, therapeutic index, patient compliance, cost efficiency, manufacturing ease, etc.

In addition, the isolation and commercial-scale preparation of a crystalline form of Compound I and corresponding pharmaceutical formulations having acceptable solid state properties (including chemical stability, thermal stability, solubility, hygroscopicity, and/or particle size), compound manufacturability (including yield, impurity rejection during crystallization, filtration properties, drying properties, and milling properties), and formulation feasibility (including stability with respect to pressure or compression forces during tableting) present a number of challenges.

Accordingly, there is a current need for one or more crystalline forms of Compound I that have an acceptable balance of these properties and can be used in the preparation of pharmaceutically acceptable solid dosage forms.

### BRIEF SUMMARY OF THE INVENTION

In one aspect, the present disclosure relates to a crystalline form of {2-[3-cyclohexyl-3-(trans-4-propoxy-cyclohexyl)-ureido]-thiazol-5-ylsulfanyl}-acetic acid. In one aspect, the crystalline form is anhydrous. In another aspect, the crystalline form is solvated.

In one aspect, the present disclosure relates to crystalline form of {2-[3-cyclohexyl-3-(trans-4-propoxy-cyclohexyl)-ureido]-thiazol-5-ylsulfanyl}-acetic acid of Formula (I)

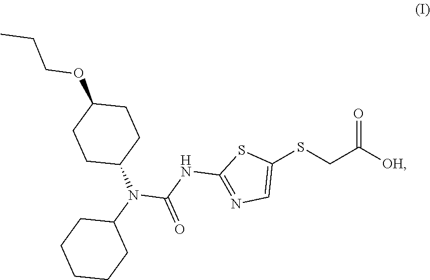 [SMILES: CCCO[C@H]1CC[C@H](N(C(=O)Nc2ncc(SCC(=O)O)s2)C2CCCCC2)CC1]

selected from the group consisting of:
   a) a crystalline form characterized by an XRPD pattern having peaks at 16.9±0.2, 17.4±0.2, and 20.1±0.2 degrees two theta;
   b) a crystalline form characterized by an XRPD pattern having peaks at 11.0±0.2, 11.6±0.2, and 17.8±0.2 degrees two theta;
   c) a crystalline form characterized by an XRPD pattern having peaks at 4.3±0.2, 17.4±0.2, and 21.6±0.2 degrees two theta;
   d) a crystalline form characterized by an XRPD pattern having peaks at 5.3±0.2, 8.7±0.2, and 26.4±0.2 degrees two theta;
   e) a crystalline form characterized by an XRPD pattern having peaks at 5.8±0.2, 17.9±0.2, and 18.9±0.2 degrees two theta;
   f) a crystalline form characterized by an XRPD pattern having peaks at 3.8±0.2, 9.5±0.2, and 16.8±0.2 degrees two theta;
   g) a crystalline form characterized by an XRPD pattern having peaks at 3.4±0.2, 21.2±0.2, and 21.9±0.2 degrees two theta;
   h) a crystalline form characterized by an XRPD pattern having peaks at 3.8±0.2, 5.3±0.2, and 8.5±0.2 degrees two theta;
   i) a crystalline form characterized by an XRPD pattern having peaks at 5.0±0.2, 16.8±0.2, and 18.8±0.2 degrees two theta; and
   j) a crystalline form characterized by an XRPD pattern having peaks at 5.9±0.2, 17.4±0.2, and 18.8±0.2 degrees two theta.

In one aspect, the present disclosure relates to a crystalline form of {2-[3-cyclohexyl-3-(trans-4-propoxy-cyclohexyl)-ureido]-thiazol-5-ylsulfanyl}-acetic acid, characterized by an XRPD pattern having peaks at 16.9±0.2, 17.4±0.2, and 20.1±0.2 degrees two theta. In one aspect, the crystalline form of {2-[3-cyclohexyl-3-(trans-4-propoxy-cyclohexyl)-ureido]-thiazol-5-ylsulfanyl}-acetic acid is characterized by an endothermic peak with onset at about 160 °C, as determined by DSC.

In one aspect, the crystalline form of {2-[3-cyclohexyl-3-(trans-4-propoxy-cyclohexyl)-ureido]-thiazol-5-ylsulfanyl}-acetic acid is characterized by an IR pattern having peaks at 1099.7±2.0, 1158.0±2.0, and 1313.2±2.0 cm⁻¹.

In one aspect, the crystalline form of {2-[3-cyclohexyl-3-(trans-4-propoxy-cyclohexyl)-ureido]-thiazol-5-ylsulfanyl}-acetic acid is characterized by a ¹³C solid state NMR substantially as shown in FIG. 4.

In one aspect, the crystalline form of {2-[3-cyclohexyl-3-(trans-4-propoxy-cyclohexyl)-ureido]-thiazol-5-ylsulfanyl}-acetic acid has a unit cell that indexes as primitive monoclinic. In another aspect, the crystalline form of {2-[3-cyclohexyl-3-(trans-4-propoxy-cyclohexyl)-ureido]-thiazol-5-ylsulfanyl}-acetic acid has a unit cell with an a value of about 10.193 Å, a b value of about 12.256 Å, and a c value of about 18.991 Å. In another aspect, the crystalline form of {2-[3-cyclohexyl-3-(trans-4-propoxy-cyclohexyl)-ureido]-thiazol-5-ylsulfanyl}-acetic acid has a unit cell with a volume of about 2370.9 Å³.

In one aspect, the crystalline form of {2-[3-cyclohexyl-3-(trans-4-propoxy-cyclohexyl)-ureido]-thiazol-5-ylsulfanyl}-acetic acid is Form A.

In one aspect, the present disclosure relates to a crystalline form of {2-[3-cyclohexyl-3-(trans-4-propoxy-cyclohexyl)-ureido]-thiazol-5-ylsulfanyl}-acetic acid, characterized by an XRPD pattern having peaks at 11.0±0.2, 11.6±0.2, and 17.8±0.2 degrees two theta. In one aspect, the crystalline form of {2-[3-cyclohexyl-3-(trans-4-propoxy-cyclohexyl)-ureido]-thiazol-5-ylsulfanyl}-acetic acid is characterized by an endothermic peak with onset at about 166 °C, as determined by DSC.

In one aspect, the crystalline form of {2-[3-cyclohexyl-3-(trans-4-propoxy-cyclohexyl)-ureido]-thiazol-5-ylsulfanyl}-acetic acid is characterized by an IR pattern having peaks at 1310.1 2.0, 1514.4±2.0, and 1661.3±2.0 cm⁻¹.

In one aspect, the crystalline form of {2-[3-cyclohexyl-3-(trans-4-propoxy-cyclohexyl)-ureido]-thiazol-5-ylsulfanyl}-acetic acid is characterized by a ¹³C solid state NMR substantially as shown in FIG. 8.

In one aspect, the crystalline form of {2-[3-cyclohexyl-3-(trans-4-propoxy-cyclohexyl)-ureido]-thiazol-5-ylsulfanyl}-acetic acid has a unit cell that indexes as primitive monoclinic. In another aspect, the crystalline form of {2-[3-cyclohexyl-3-(trans-4-propoxy-cyclohexyl)-ureido]-thiazol-5-ylsulfanyl}-acetic acid has a unit cell with an a value of about 11.028 Å, a b value of about 11.933 Å, and a c value of about 18.737 Å. In another aspect, the crystalline form of {2-[3-cyclohexyl-3-(trans-4-propoxy-cyclohexyl)-ureido]-thiazol-5-ylsulfanyl}-acetic acid has a unit cell with a volume of about 2449.0 Å³.

In one aspect, the crystalline form of {2-[3-cyclohexyl-3-(trans-4-propoxy-cyclohexyl)-ureido]-thiazol-5-ylsulfanyl}-acetic acid is Form B.

In one aspect, the present disclosure relates to a crystalline form of {2-[3-cyclohexyl-3-(trans-4-propoxy-cyclohexyl)-ureido]-thiazol-5-ylsulfanyl}-acetic acid, characterized by an XRPD pattern having peaks at 4.3±0.2, 17.4±0.2, and 21.6±0.2 degrees two theta.

In one aspect, the crystalline form of {2-[3-cyclohexyl-3-(trans-4-propoxy-cyclohexyl)-ureido]-thiazol-5-ylsulfanyl}-acetic acid is characterized by an endothermic peak with onset at about 149 °C, as determined by DSC.

In one aspect, the crystalline form of {2-[3-cyclohexyl-3-(trans-4-propoxy-cyclohexyl)-ureido]-thiazol-5-ylsulfanyl}-acetic acid is a dichloromethane solvate.

In one aspect, the crystalline form of {2-[3-cyclohexyl-3-(trans-4-propoxy-cyclohexyl)-ureido]-thiazol-5-ylsulfanyl}-acetic acid has a unit cell that indexes as primitive monoclinic. In another aspect, the crystalline form of {2-[3-cyclohexyl-3-(trans-4-propoxy-cyclohexyl)-ureido]-thiazol-5-ylsulfanyl}-acetic acid has a unit cell with an a value of about 5.541 Å, a b value of about 13.040 Å, and a c value of about 40.818 Å. In another aspect, the crystalline form of {2-[3-cyclohexyl-3-(trans-4-propoxy-cyclohexyl)-ureido]-thiazol-5-ylsulfanyl}-acetic acid has a unit cell with a volume of about 2947.6 Å³.

In one aspect, the crystalline form of {2-[3-cyclohexyl-3-(trans-4-propoxy-cyclohexyl)-ureido]-thiazol-5-ylsulfanyl}-acetic acid is Form C.

In one aspect, the present disclosure relates to a crystalline form of {2-[3-cyclohexyl-3-(trans-4-propoxy-cyclohexyl)-ureido]-thiazol-5-ylsulfanyl}-acetic acid, characterized by an XRPD pattern having peaks at 5.3±0.2, 8.7±0.2, and 26.4±0.2 degrees two theta.

In one aspect, the crystalline form of {2-[3-cyclohexyl-3-(trans-4-propoxy-cyclohexyl)-ureido]-thiazol-5-ylsulfanyl}-acetic acid is characterized by an endothermic peak with onset at about 147 °C, as determined by DSC.

In one aspect, the crystalline form of {2-[3-cyclohexyl-3-(trans-4-propoxy-cyclohexyl)-ureido]-thiazol-5-ylsulfanyl}-acetic acid is characterized by a ¹³C solid state NMR substantially as shown in FIG. 13.

In one aspect, the crystalline form of {2-[3-cyclohexyl-3-(trans-4-propoxy-cyclohexyl)-ureido]-thiazol-5-ylsulfanyl}-acetic acid is Form D.

In one aspect, the present disclosure relates to a crystalline form of {2-[3-cyclohexyl-3-(trans-4-propoxy-cyclohexyl)-ureido]-thiazol-5-ylsulfanyl}-acetic acid, characterized by an XRPD pattern having peaks at 5.8±0.2, 17.9±0.2, and 18.9±0.2 degrees two theta.

In one aspect, the crystalline form of {2-[3-cyclohexyl-3-(trans-4-propoxy-cyclohexyl)-ureido]-thiazol-5-ylsulfanyl}-acetic acid is characterized by an endothermic peak with onset at about 171 °C, as determined by DSC.

In one aspect, the crystalline form of {2-[3-cyclohexyl-3-(trans-4-propoxy-cyclohexyl)-ureido]-thiazol-5-ylsulfanyl}-acetic acid is Form E.

In one aspect, the present disclosure relates to a crystalline form of {2-[3-cyclohexyl-3-(trans-4-propoxy-cyclohexyl)-ureido]-thiazol-5-ylsulfanyl}-acetic acid, characterized by an XRPD pattern having peaks at 3.8±0.2, 9.5±0.2, and 16.8±0.2 degrees two theta.

In one aspect, the crystalline form of {2-[3-cyclohexyl-3-(trans-4-propoxy-cyclohexyl)-ureido]-thiazol-5-ylsulfanyl}-acetic acid is Form F.

In one aspect, the present disclosure relates to a crystalline form of {2-[3-cyclohexyl-3-(trans-4-propoxy-cyclohexyl)-ureido]-thiazol-5-ylsulfanyl}-acetic acid, characterized by an XRPD pattern having peaks at 3.4±0.2, 21.2±0.2, and 21.9±0.2 degrees two theta.

In one aspect, the crystalline form of {2-[3-cyclohexyl-3-(trans-4-propoxy-cyclohexyl)-ureido]-thiazol-5-ylsulfanyl}-acetic acid is Form G.

In one aspect, the present disclosure relates to a crystalline form of {2-[3-cyclohexyl-3-(trans-4-propoxy-cyclohexyl)-ureido]-thiazol-5-ylsulfanyl}-acetic acid, characterized by an XRPD pattern having peaks at 3.8±0.2, 5.3±0.2, and 8.5±0.2 degrees two theta.

In one aspect, the crystalline form of {2-[3-cyclohexyl-3-(trans-4-propoxy-cyclohexyl)-ureido]-thiazol-5-ylsulfanyl}-acetic acid is Form H.

In one aspect, the present disclosure relates to a crystalline form of {2-[3-cyclohexyl-3-(trans-4-propoxy-cyclohexyl)-ureido]-thiazol-5-ylsulfanyl}-acetic acid, characterized by an XRPD pattern having peaks at 5.0±0.2, 16.8±0.2, and 18.8±0.2 degrees two theta.

In one aspect, the crystalline form of {2-[3-cyclohexyl-3-(trans-4-propoxy-cyclohexyl)-ureido]-thiazol-5-ylsulfanyl}-acetic acid is Form I.

In one aspect, the present disclosure relates to a crystalline form of {2-[3-cyclohexyl-3-(trans-4-propoxy-cyclohexyl)-ureido]-thiazol-5-ylsulfanyl}-acetic acid, characterized by an XRPD pattern having peaks at 5.9±0.2, 17.4±0.2, and 18.8±0.2 degrees two theta.

In one aspect, the crystalline form of {2-[3-cyclohexyl-3-(trans-4-propoxy-cyclohexyl)-ureido]-thiazol-5-ylsulfanyl}-acetic acid is characterized by an endothermic peak with onset at about 164 °C, as determined by DSC.

In one aspect, the crystalline form of {2-[3-cyclohexyl-3-(trans-4-propoxy-cyclohexyl)-ureido]-thiazol-5-ylsulfanyl}-acetic acid is Form J.

In some aspects, the crystalline form is substantially free of other polymorphic forms. In some aspects, the crystalline form has a polymorphic purity of at least about 80%.

In one aspect, the crystalline form of {2-[3-cyclohexyl-3-(trans-4-propoxy-cyclohexyl)-ureido]-thiazol-5-ylsulfanyl}-acetic acid is selected from the group consisting of Form A, Form B, Form C, Form D, Form E, Form F, Form G, Form H, Form I, and Form J. In one aspect, the crystalline form of {2-[3-cyclohexyl-3-(trans-4-propoxy-cyclohexyl)-ureido]-thiazol-5-ylsulfanyl}-acetic acid is Form A.

In some aspects, the present disclosure relates to a pharmaceutical composition comprising any one or more of the crystalline forms discussed above and a pharmaceutically acceptable carrier, diluent, excipient, or a mixture thereof.

In some aspects, the present disclosure relates to a method of treating a type of diabetes mellitus or other disorders, where the method comprises administering the pharmaceutical composition discussed above to a patient in need thereof. In some aspects, the type of diabetes mellitus is type 1 diabetes. In some aspects, the type of diabetes mellitus is type 2 diabetes.

In some aspects, the pharmaceutical composition is administered orally. In some aspects, the pharmaceutical composition is administered as a tablet. In some aspects, the patient is administered up to about 2000 mg of {2-[3-cyclohexyl-3-(trans-4-propoxy-cyclohexyl)-ureido]-thiazol-5-ylsulfanyl}-acetic acid daily.

In some aspects, the present disclosure provides methods of making a crystalline form of {2-[3-cyclohexyl-3-(trans-4-propoxy-cyclohexyl)-ureido]-thiazol-5-ylsulfanyl}-acetic acid where the crystalline form is selected from the group consisting of Form A, Form B, Form C, Form D, Form E, Form F, Form G, Form H, Form I, and Form J.

### BRIEF DESCRIPTION OF THE FIGURES

FIG. 1 is a powder X-ray diffraction pattern (“XRPD”) corresponding to crystalline Form A.

FIG. 2A is a differential scanning calorimetry thermogram (“DSC”) corresponding to crystalline Form A.

FIG. 2B is a thermogravimetric analysis thermogram (“TGA”) corresponding to crystalline Form A.

FIG. 3 is an Infrared (“IR”) spectrum corresponding to crystalline Form A.

FIG. 4 is a ¹³C solid state NMR corresponding to crystalline Form A.

FIG. 5 is an XRPD corresponding to crystalline Form B.

FIG. 6A is a DSC corresponding to crystalline Form B.

FIG. 6B is a TGA corresponding to crystalline Form B.

FIG. 7 is an IR spectrum corresponding to crystalline Form B.

FIG. 8 is a ¹³C solid state NMR corresponding to crystalline Form B.

FIG. 9 is an XRPD corresponding to crystalline Form C.

FIG. 10A is a DSC corresponding to crystalline Form C.

FIG. 10B is a TGA corresponding to crystalline Form C.

FIG. 11 is an XRPD corresponding to crystalline Form D.

FIG. 12A is a DSC corresponding to crystalline Form D.

FIG. 12B is a TGA corresponding to crystalline Form D.

FIG. 13 is a ¹³C solid state NMR corresponding to crystalline Form D.

FIG. 14 is an XRPD corresponding to crystalline Form E.

FIG. 15A is a DSC corresponding to crystalline Form E.

FIG. 15B is a TGA corresponding to crystalline Form E.

FIG. 16 is an XRPD corresponding to crystalline Form F.

FIG. 17 is an XRPD corresponding to crystalline Form G.

FIG. 18 is an XRPD corresponding to crystalline Form H.

FIG. 19 is an XRPD corresponding to crystalline Form I.

FIG. 20 is an XRPD corresponding to crystalline Form J.

FIG. 21A is a DSC corresponding to crystalline Form J.

FIG. 21B is a TGA corresponding to crystalline Form J.

### DETAILED DESCRIPTION OF THE INVENTION

### I. Definitions

To facilitate understanding of the disclosure set forth herein, a number of terms are defined below.

Generally, the nomenclature used herein and the laboratory procedures in organic chemistry, medicinal chemistry, and pharmacology described herein are those well-known and commonly employed in the art. Unless defined otherwise, all technical and scientific terms used herein generally have the same meaning as commonly understood by one of ordinary skill in the art to which this disclosure belongs.

In this specification and the appended claims, the singular forms “a,” “an” and “the” include plural referents unless the context clearly dictates otherwise. The terms “a” (or “an”), as well as the terms “one or more,” and “at least one” can be used interchangeably herein. In certain aspects, the term “a” or “an” means “single.” In other aspects, the term “a” or “an” includes “two or more” or “multiple.”

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

The term “Compound I” refers to the chemical compound {2-[3-cyclohexyl-3-(trans-4-propoxy-cyclohexyl)-ureido]-thiazol-5-ylsulfanyl}-acetic acid.

The term “subject” refers to an animal, including, but not limited to, a primate (e.g., human), cow, sheep, goat, horse, dog, cat, rabbit, rat, or mouse. The terms “subject” and “patient” are used interchangeably herein in reference, for example, to a mammalian subject, such as a human subject.

The terms “treat,” “treating,” and “treatment” are meant to include alleviating or abrogating a disorder, disease, or condition, or one or more of the symptoms associated with the disorder, disease, or condition; or alleviating the cause(s) of the disorder, disease, or condition itself.

The terms “pharmaceutically acceptable carrier,” “pharmaceutically acceptable diluent,” or “pharmaceutically acceptable excipient,” refer to a pharmaceutically-acceptable material, composition, or vehicle, such as a liquid or solid filler, diluent, excipient, solvent, or encapsulating material. In one aspect, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See Remington: The Science and Practice of Pharmacy, 21st Edition, Lippincott Williams & Wilkins: Philadelphia, PA, 2005; Handbook of Pharmaceutical Excipients, 5th Edition, Rowe et al., Eds., The Pharmaceutical Press and the American Pharmaceutical Association: 2005; and Handbook of Pharmaceutical Additives, 3rd Edition, Ash and Ash Eds., Gower Publishing Company: 2007; Pharmaceutical Preformulation and Formulation, Gibson Ed., CRC Press LLC: Boca Raton, FL, 2004 (incorporated herein by reference).

The terms “about” or “approximately” means an acceptable error for a particular value as determined by one of ordinary skill in the art, which depends in part on how the value is measured or determined. In certain aspects, the term “about” or “approximately” means within 1, 2, 3, or 4 standard deviations. In certain embodiments, the term “about” or “approximately” means within 50%, 20%, 15%, 10%, 9%, 8%, 7%, 6%, 5%, 4%, 3%, 2%, 1%, 0.5%, or 0.05% of a given value or range.

The terms “active ingredient” and “active substance” refer to a compound, which is administered, alone or in combination with one or more pharmaceutically acceptable excipients, to a subject for treating, delaying onset of, or ameliorating one or more symptoms of a condition, disorder, or disease. As used herein, “active ingredient” and “active substance” may be an optically active isomer of a compound described herein.

The term “solvate” or “solvated” refers to a compound provided herein or a salt thereof, which further includes a stoichiometric or non-stoichiometric amount of solvent bound by non-covalent intermolecular forces. Where the solvent is water, the solvate is a hydrate. Where the solvent includes ethanol, the compound can be an ethanol solvate.

The term “polymorph” as used herein refers to a crystalline form of a compound or a salt, hydrate, or solvate thereof, in a particular crystal packing arrangement. All polymorphs have the same elemental composition. The term “crystalline,” as used herein, refers to a solid state form which consists of orderly arrangement of structural units. Different crystalline forms of the same compound, or a salt, hydrate, or solvate thereof, arise from different packing of the molecules in the solid state, which results in different crystal symmetries and/or unit cell parameter. Different crystalline forms usually have different X-ray diffraction patterns, infrared spectra, melting points, density, hardness, crystal shape, optical and electrical properties, stability, and solubility. See, e.g., Remington's Pharmaceutical Sciences, 18^{th} ed., Mack Publishing, Easton PA, 173 (1990); The United States Pharmacopeia, 23^{rd} ed., 1843-1844 (1995) (incorporated herein by reference).

Crystalline forms are most commonly characterized by X-ray powder diffraction (XRPD). An XRPD pattern of reflections (peaks, typically expressed in degrees 2-theta) is commonly considered a fingerprint of a particular crystalline form. The relative intensities of the XRPD peaks can widely vary depending on, inter alia, the sample preparation technique, crystal size distribution, filters, the sample mounting procedure, and the particular instrument employed. In some instances, new peaks may be observed or existing peaks may disappear, depending on the type of instrument or the settings. In some instances, any particular peak in an XRPD pattern may appear as a singlet, doublet, triplet, quartet, or multiplet, depending on the type of instrument or the settings, the sensitivity of the instrument, measuring conditions, and/or purity of the crystalline form. In some instances, any particular peak in an XRPD may appear in a symmetric shape or in an asymmetric shape, e.g., having a shoulder. Moreover, instrument variation and other factors can affect the 2-theta values. A skilled artisan understanding these variations is capable of discriminating or ascertaining the defining features or characteristics of a particular crystal form using XRPD, as well as using other known physicochemical techniques.

The term “anhydrate” or “anhydrous” as applied to a compound refers to a solid state wherein the compound contains no structural water within the crystal lattice.

Unless the context requires otherwise, the terms “comprise,” “comprises,” and “comprising” are used on the basis and clear understanding that they are to be interpreted inclusively, rather than exclusively, and that Applicant intends each of those words to be so interpreted in construing this patent, including the claims below.

For all embodiments disclosed herein, a peak positional reproducibility is associated with the values of degree-2θ (XRPD), ppm (¹³C solid state NMR), and cm⁻¹ (IR). Accordingly, it will be understood that all peaks disclosed herein have the value disclosed±the peak positional reproducibility associated with each analytical technique. The XRPD peak positional reproducibility is ±0.2 expressed in degree-2θ. The ¹³C NMR peak positional reproducibility is ±0.2 ppm. The IR peak positional reproducibility is ±2 cm⁻¹.

Unless defined otherwise, all technical and scientific terms used herein have the same meaning as commonly understood by one of ordinary skill in the art to which this disclosure belongs. In case of conflict, the present application including the definitions will control. Unless otherwise required by context, singular terms shall include pluralities and plural terms shall include the singular. All publications, patents and other references mentioned herein are incorporated by reference in their entireties for all purposes as if each individual publication or patent application were specifically and individually indicated to be incorporated by reference.

### II. Crystalline Forms

In one aspect, the present disclosure relates to a crystalline form of {2-[3-cyclohexyl-3-(trans-4-propoxy-cyclohexyl)-ureido]-thiazol-5-ylsulfanyl}-acetic acid. In one aspect, the crystalline form is anhydrous as determined by ¹H NMR. In another aspect, the crystalline form is solvated as determined by ¹H NMR.

In one aspect, the present disclosure relates to crystalline form of {2-[3-cyclohexyl-3-(trans-4-propoxy-cyclohexyl)-ureido]-thiazol-5-ylsulfanyl}-acetic acid of Formula (I)

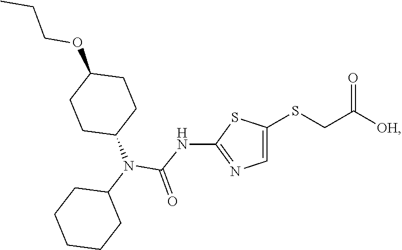 [SMILES: CCCO[C@H]1CC[C@H](N(C(=O)Nc2ncc(SCC(=O)O)s2)C2CCCCC2)CC1]

selected from the group consisting of
   a) a crystalline form characterized by an XRPD pattern having peaks at 16.9±0.2, 17.4±0.2, and 20.1±0.2 degrees two theta;
   b) a crystalline form characterized by an XRPD pattern having peaks at 11.0±0.2, 11.6±0.2, and 17.8±0.2 degrees two theta;
   c) a crystalline form characterized by an XRPD pattern having peaks at 4.3 0.2, 17.4±0.2, and 21.6±0.2 degrees two theta;
   d) a crystalline form characterized by an XRPD pattern having peaks at 5.3±0.2, 8.7±0.2, and 26.4±0.2 degrees two theta;
   e) a crystalline form characterized by an XRPD pattern having peaks at 5.8±0.2, 17.9±0.2, and 18.9±0.2 degrees two theta;
   f) a crystalline form characterized by an XRPD pattern having peaks at 3.8±0.2, 9.5±0.2, and 16.8±0.2 degrees two theta;
   g) a crystalline form characterized by an XRPD pattern having peaks at 3.4±0.2, 21.2±0.2, and 21.9±0.2 degrees two theta;
   h) a crystalline form characterized by an XRPD pattern having peaks at 3.8±0.2, 5.3±0.2, and 8.5±0.2 degrees two theta;
   i) a crystalline form characterized by an XRPD pattern having peaks at 5.0±0.2, 16.8±0.2, and 18.8±0.2 degrees two theta; and
   j) a crystalline form characterized by an XRPD pattern having peaks at 5.9±0.2, 17.4±0.2, and 18.8±0.2 degrees two theta.

### A. Crystalline Form A

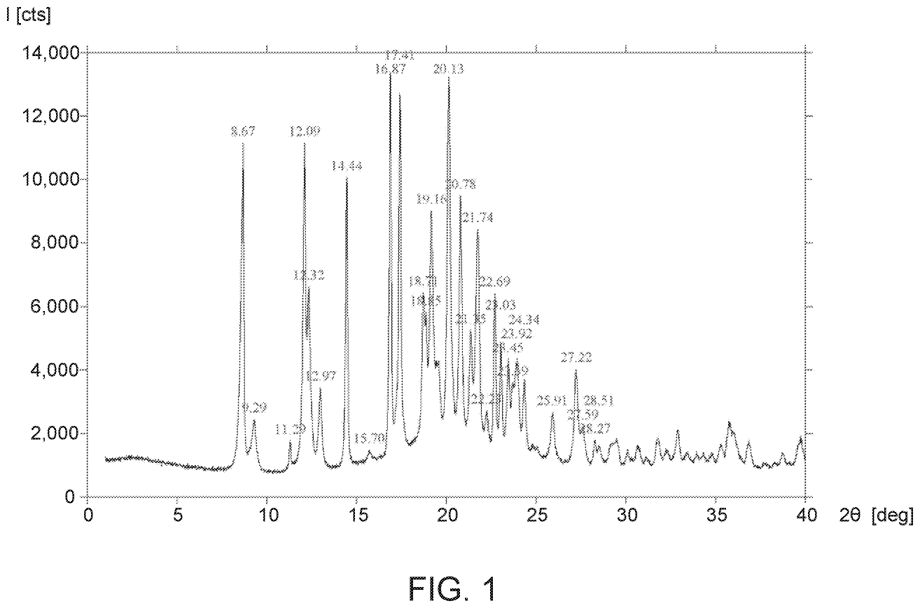

In one aspect, the present disclosure relates to a crystalline form of Compound I, characterized by an XRPD pattern having peaks at 16.9±0.2, 17.4±0.2, and 20.1±0.2 degrees two theta. In one aspect, the present disclosure relates to a crystalline form of Compound I, characterized by an XRPD pattern having peaks at 8.7±0.2, 16.9±0.2, 17.4±0.2, and 20.1±0.2 degrees two theta. In one aspect, the crystalline form of Compound I is characterized by an XRPD pattern substantially as shown in FIG. 1.

In one aspect, the crystalline form of Compound I is characterized by the following XRPD pattern in Table 1 expressed in terms of the degree 2θ and relative intensities:

TABLE 1

| Angle | Relative |
|---|---|
| (Degree 2θ) | Intensity * (%) |
| 8.7 ± 0.2 | 83 |
| 9.3 ± 0.2 | 18 |
| 11.3 ± 0.2 | 13 |
| 12.1 ± 0.2 | 83 |
| 12.3 ± 0.2 | 49 |
| 13.0 ± 0.2 | 26 |
| 14.4 ± 0.2 | 76 |
| 15.7 ± 0.2 | 11 |
| 16.9 ± 0.2 | 100 |
| 17.4 ± 0.2 | 95 |
| 18.7 ± 0.2 | 48 |
| 18.9 ± 0.2 | 43 |
| 19.2 ± 0.2 | 68 |
| 20.1 ± 0.2 | 99 |
| 20.8 ± 0.2 | 71 |
| 21.4 ± 0.2 | 39 |
| 21.7 ± 0.2 | 63 |
| 22.2 ± 0.2 | 20 |
| 22.7 ± 0.2 | 48 |
| 23.0 ± 0.2 | 36 |
| 23.5 ± 0.2 | 33 |
| 23.7 ± 0.2 | 27 |
| 23.9 ± 0.2 | 31 |
| 24.3 ± 0.2 | 28 |
| 25.9 ± 0.2 | 20 |
| 27.2 ± 0.2 | 30 |
| 27.6 ± 0.2 | 17 |
| 28.3 ± 0.2 | 13 |
| 28.5 ± 0.2 | 12 |

* The relative intensities can change depending on the crystal size and morphology.

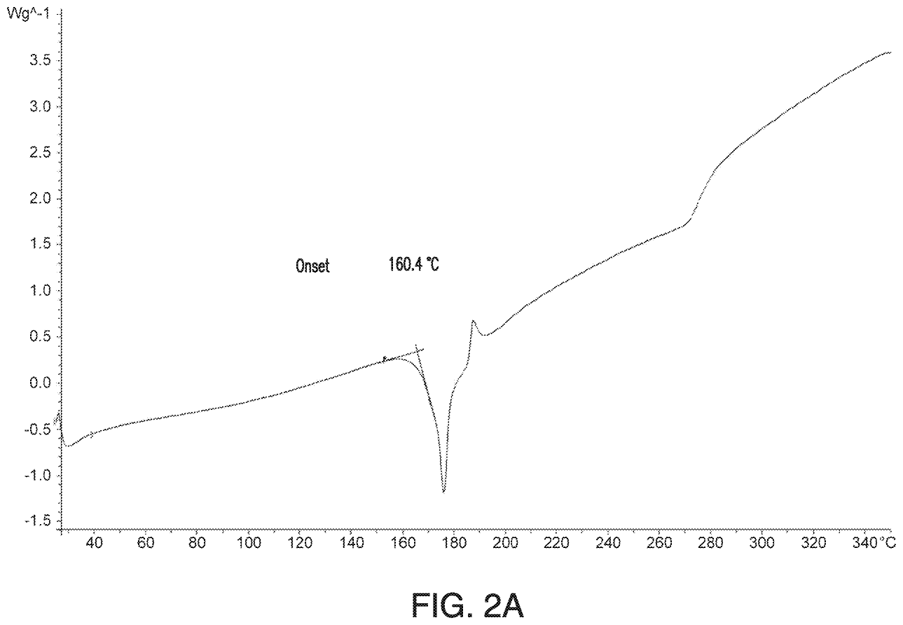

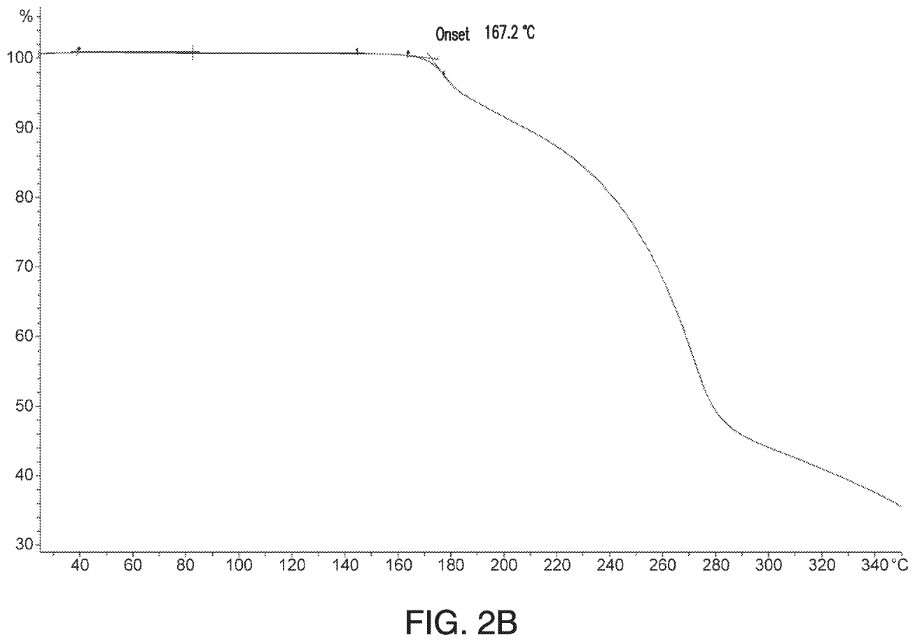

In one aspect, the crystalline form of Compound I is characterized by an endothermic peak with onset at about 160 °C, as determined by DSC. In one aspect, the crystalline form of Compound I is characterized by a DSC profile substantially as shown in FIG. 2A. In one aspect, the crystalline form of Compound I is characterized by a TGA profile substantially as shown in FIG. 2B.

In one aspect, the crystalline form of Compound I is characterized by an IR pattern having peaks at 1099.7±2.0, 1158.0±2.0, and 1313.2±2.0 cm⁻¹. In one aspect, the crystalline form of Compound I is characterized by an IR pattern having peaks at 1099.7±2.0, 1158.0±2.0, 1238.7±2.0, and 1313.2±2.0 cm⁻¹. In one aspect, the crystalline form of Compound I is characterized by the following IR peaks in Table 2.

TABLE 2

| Position (cm⁻¹) | Log(1/R) |
|---|---|
| 713.1 | 0.0120 |
| 723.0 | 0.0108 |
| 747.5 | 0.0083 |
| 777.1 | 0.0053 |
| 790.1 | 0.0063 |
| 807.0 | 0.0094 |
| 857.1 | 0.0116 |
| 893.9 | 0.0125 |
| 913.1 | 0.0114 |
| 941.2 | 0.0054 |
| 950.9 | 0.0054 |
| 1004.4 | 0.0108 |
| 1027.9 | 0.0114 |
| 1048.2 | 0.0122 |
| 1099.7 | 0.0316 |
| 1141.7 | 0.0152 |
| 1158.0 | 0.0284 |
| 1178.0 | 0.0127 |
| 1208.0 | 0.0146 |
| 1238.7 | 0.0256 |
| 1263.6 | 0.0134 |
| 1313.2 | 0.0440 |
| 1346.4 | 0.0247 |
| 1359.6 | 0.0234 |
| 1398.8 | 0.0132 |
| 1449.9 | 0.0131 |
| 1498.7 | 0.0136 |
| 1544.2 | 0.0282 |
| 1646.7 | 0.0265 |
| 1662.4 | 0.0402 |
| 1698.9 | 0.0109 |
| 1884.3 | 0.0034 |
| 2859.5 | 0.0067 |
| 2931.3 | 0.0109 |
| 3180.1 | 0.0016 |
| 3224.6 | 0.0019 |

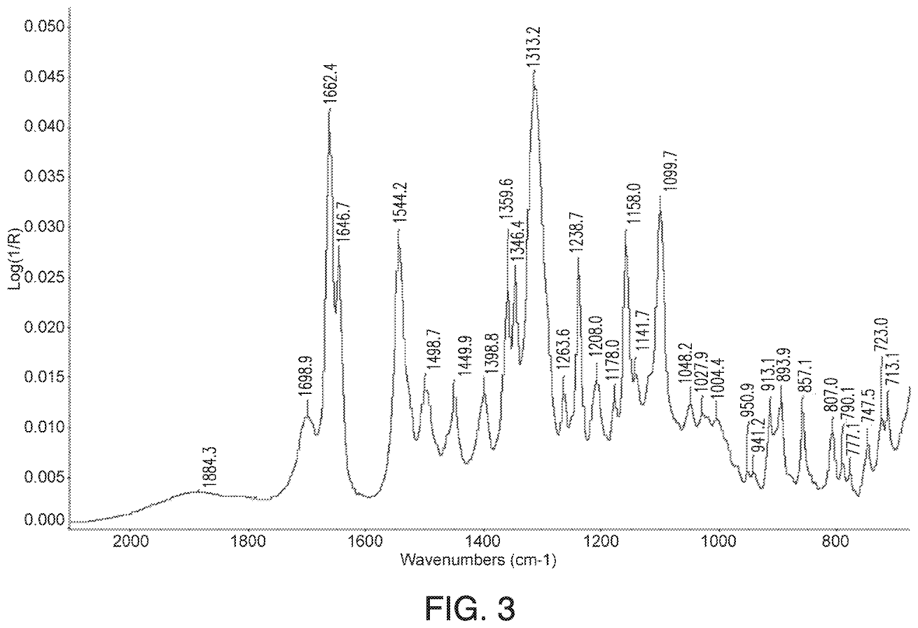

In one aspect, the crystalline form of Compound I is characterized by an IR pattern substantially as shown in FIG. 3.

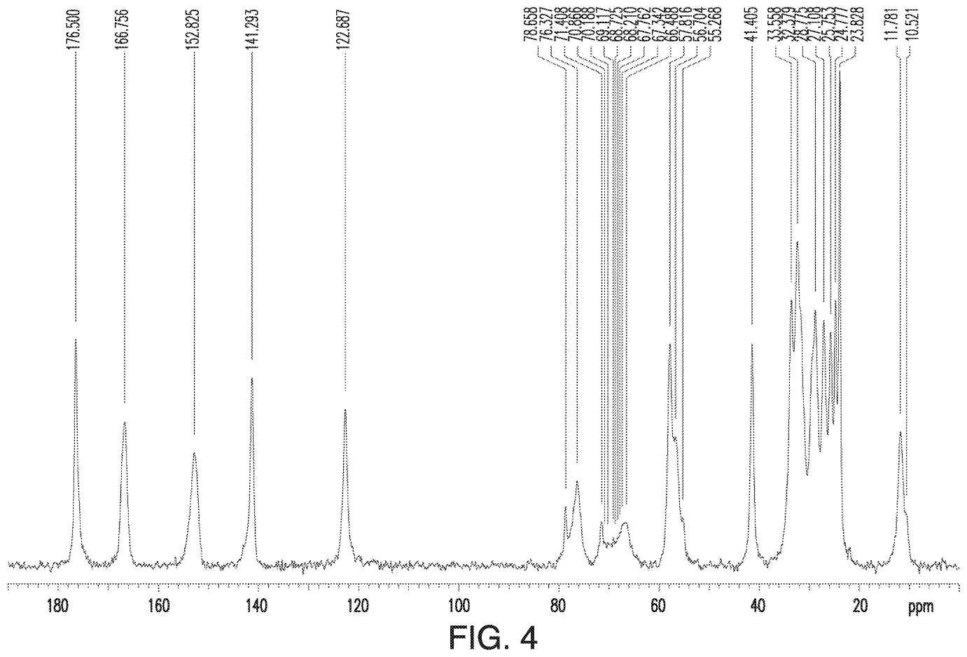

In one aspect, the crystalline form of Compound I is characterized by a ¹³C solid state NMR substantially as shown in FIG. 4.

In one aspect, the crystalline form is anhydrous as determined by ¹H NMR.

In one aspect, the crystalline form of Compound I has a unit cell that indexes as primitive monoclinic. In another aspect, the crystalline form of Compound I has a unit cell with an a value of about 10.193 Å, a b value of about 12.256 Å, and a c value of about 18.991 Å. In another aspect, the crystalline form of Compound I has a unit cell with a volume of about 2370.9 Å³.

In one aspect, the crystalline form of Compound I is Form A.

### B. Crystalline Form B

In one aspect, the present disclosure relates to a crystalline form of Compound I, characterized by an XRPD pattern having peaks at 11.0±0.2, 11.6±0.2, and 17.8±0.2 degrees two theta. In one aspect, the present disclosure relates to a crystalline form of Compound I, characterized by an XRPD pattern having peaks at 11.0±0.2, 11.6±0.2, 17.8±0.2, and 21.1±0.2 degrees two theta.

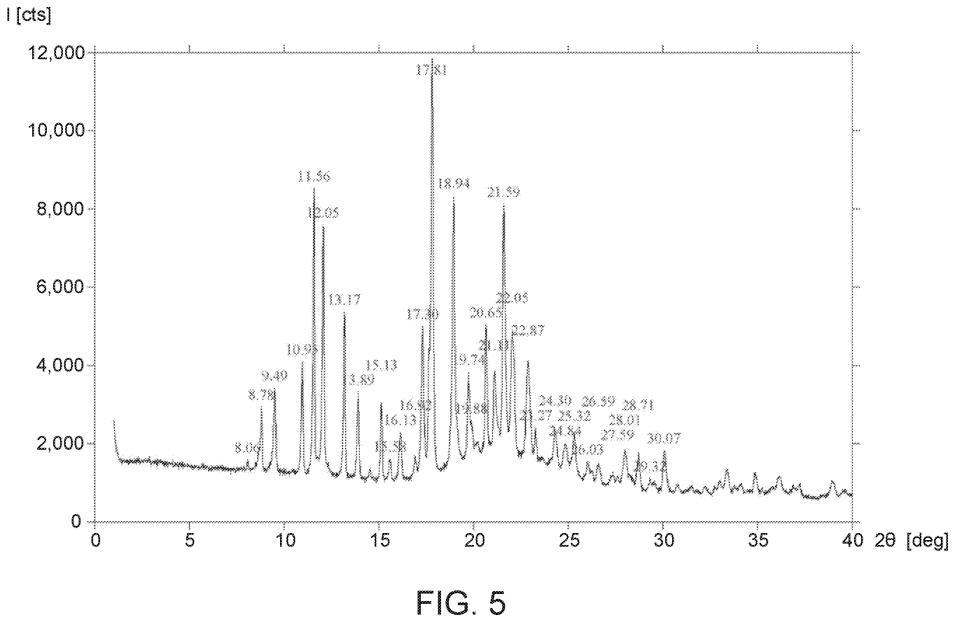

In one aspect, the crystalline form of Compound I is characterized by an XRPD pattern substantially as shown in FIG. 5.

In one aspect, the crystalline form of Compound I is characterized by the following XRPD pattern in Table 3 expressed in terms of the degree 2θ and relative intensities:

TABLE 3

| Angle | Relative |
|---|---|
| (Degree 2θ) | Intensity * (%) |
| 8.1 ± 0.2 | 13 |
| 8.8 ± 0.2 | 24 |
| 9.5 ± 0.2 | 29 |
| 11 ± 0.2 | 34 |
| 11.6 ± 0.2 | 71 |
| 12.1 ± 0.2 | 64 |
| 13.2 ± 0.2 | 45 |
| 13.9 ± 0.2 | 28 |
| 15.1 ± 0.2 | 26 |
| 15.6 ± 0.2 | 13 |
| 16.1 ± 0.2 | 19 |
| 16.9 ± 0.2 | 14 |
| 17.3 ± 0.2 | 42 |
| 17.8 ± 0.2 | 100 |
| 18.9 ± 0.2 | 70 |
| 19.7 ± 0.2 | 32 |
| 19.9 ± 0.2 | 21 |
| 20.7 ± 0.2 | 42 |
| 21.1 ± 0.2 | 33 |
| 21.6 ± 0.2 | 68 |
| 22.1 ± 0.2 | 41 |
| 22.9 ± 0.2 | 35 |
| 23.3 ± 0.2 | 20 |
| 24.3 ± 0.2 | 20 |
| 24.8 ± 0.2 | 17 |
| 25.3 ± 0.2 | 19 |
| 26 ± 0.2 | 13 |
| 26.6 ± 0.2 | 13 |
| 27.6 ± 0.2 | 10 |
| 28 ± 0.2 | 15 |
| 28.7 ± 0.2 | 15 |
| 29.3 ± 0.2 | 9 |
| 30.1 ± 0.2 | 15 |

* The relative intensities can change depending on the crystal size and morphology.

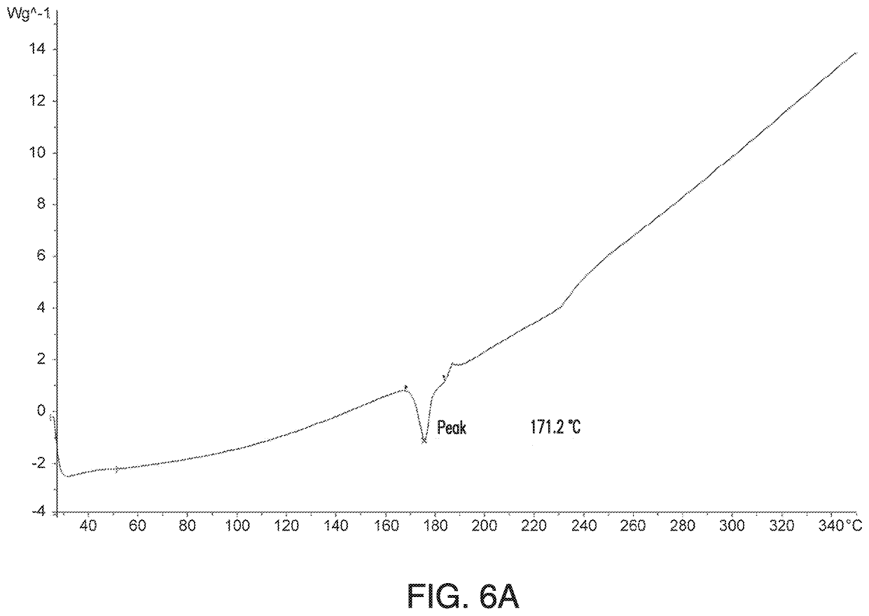

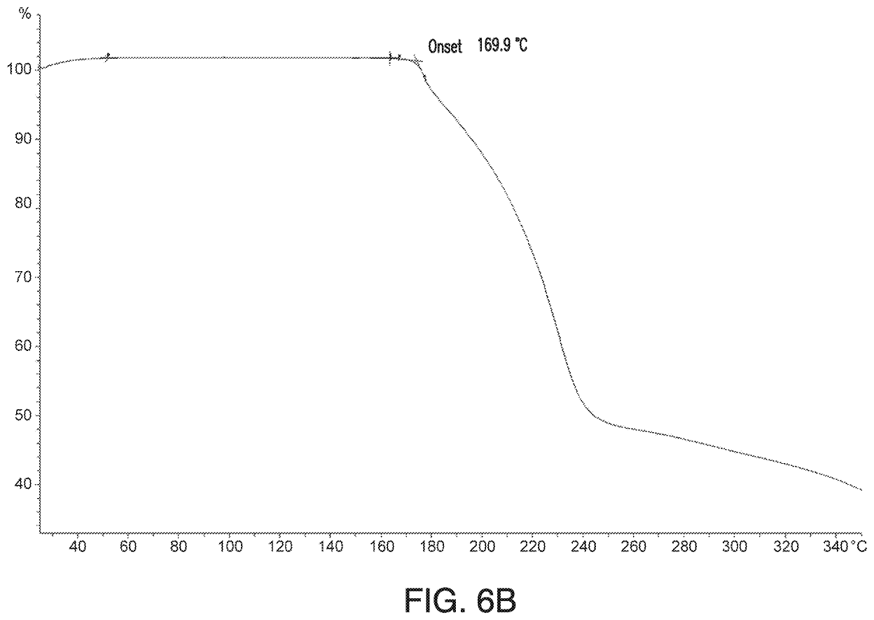

In one aspect, the crystalline form of Compound I is characterized by an endothermic peak with onset at about 166 °C, as determined by DSC. In one aspect, the crystalline form of Compound I is characterized by a DSC profile substantially as shown in FIG. 6A. In one aspect, the crystalline form of Compound I is characterized by a TGA profile substantially as shown in FIG. 6B.

In one aspect, the crystalline form of Compound I is characterized by an IR pattern having peaks at 1310.1±2.0, 1514.4±2.0, and 1661.3±2.0 cm⁻¹. In one aspect, the crystalline form of Compound I is characterized by an IR pattern having peaks at 1097.3±2.0, 1310.1 2.0, 1541.4±2.0, and 1661.3±2.0 cm⁻¹. In one aspect, the crystalline form of Compound I is characterized by the following IR peaks in Table 4.

TABLE 4

| Position (cm⁻¹) | Log(1/R) |
|---|---|
| 713.6 | 0.0109 |
| 726.3 | 0.0166 |
| 751.3 | 0.0080 |
| 775.2 | 0.0062 |
| 787.9 | 0.0099 |
| 807.3 | 0.0124 |
| 859.5 | 0.0120 |
| 894.7 | 0.0124 |
| 911.7 | 0.0146 |
| 942.3 | 0.0088 |
| 970.6 | 0.0077 |
| 999.7 | 0.0127 |
| 1018.6 | 0.0127 |
| 1049.1 | 0.0121 |
| 1097.3 | 0.0316 |
| 1122.7 | 0.0162 |
| 1137.9 | 0.0173 |
| 1159.8 | 0.0307 |
| 1183.7 | 0.0146 |
| 1208.6 | 0.0219 |
| 1239.5 | 0.0271 |
| 1265.0 | 0.0193 |
| 1310.1 | 0.0507 |
| 1356.7 | 0.0284 |
| 1399.3 | 0.0165 |
| 1451.7 | 0.0167 |
| 1500.3 | 0.0199 |
| 1541.4 | 0.0346 |
| 1661.3 | 0.0487 |
| 1697.0 | 0.0160 |
| 1886.9 | 0.0075 |
| 2858.8 | 0.0112 |
| 2932.8 | 0.0159 |
| 3184.1 | 0.0057 |
| 3229.2 | 0.0063 |

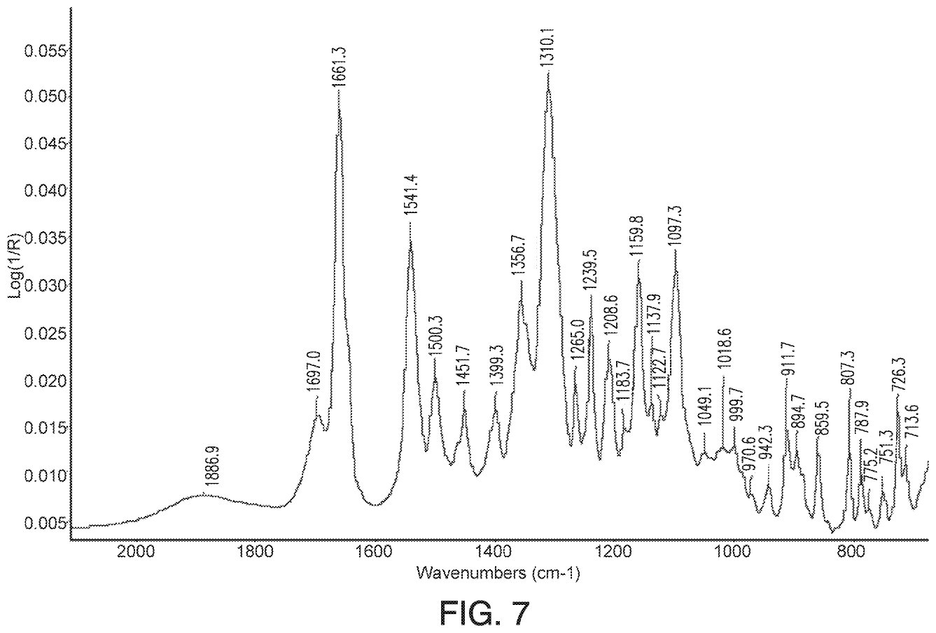

In one aspect, the crystalline form of Compound I is characterized by an IR pattern substantially as shown in FIG. 7.

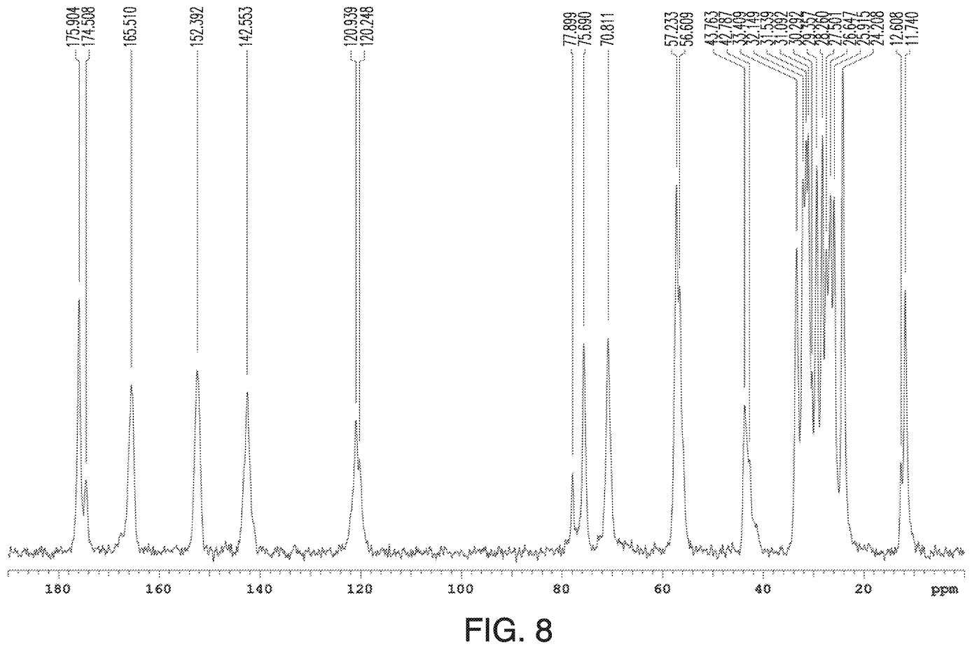

In one aspect, the crystalline form of Compound I is characterized by a ¹³C solid state NMR substantially as shown in FIG. 8.

In one aspect, the crystalline form is anhydrous as determined by ¹H NMR.

In one aspect, the crystalline form of Compound I has a unit cell that indexes as primitive monoclinic. In another aspect, the crystalline form of Compound I has a unit cell with an a value of about 11.028 Å, a b value of about 11.933 Å, and a c value of about 18.737 Å. In another aspect, the crystalline form of Compound I has a unit cell with a volume of about 2449.0 Å³.

In one aspect, the crystalline form of Compound I is Form B.

### C. Crystalline Form C

In one aspect, the present disclosure relates to a crystalline form of Compound I, characterized by an XRPD pattern having peaks at 4.3±0.2, 17.4±0.2, and 21.6±0.2 degrees two theta. In one aspect, the present disclosure relates to a crystalline form of Compound I, characterized by an XRPD pattern having peaks at 4.3±0.2, 8.0±0.2, 17.4±0.2, and about 21.6±0.2 degrees two theta.

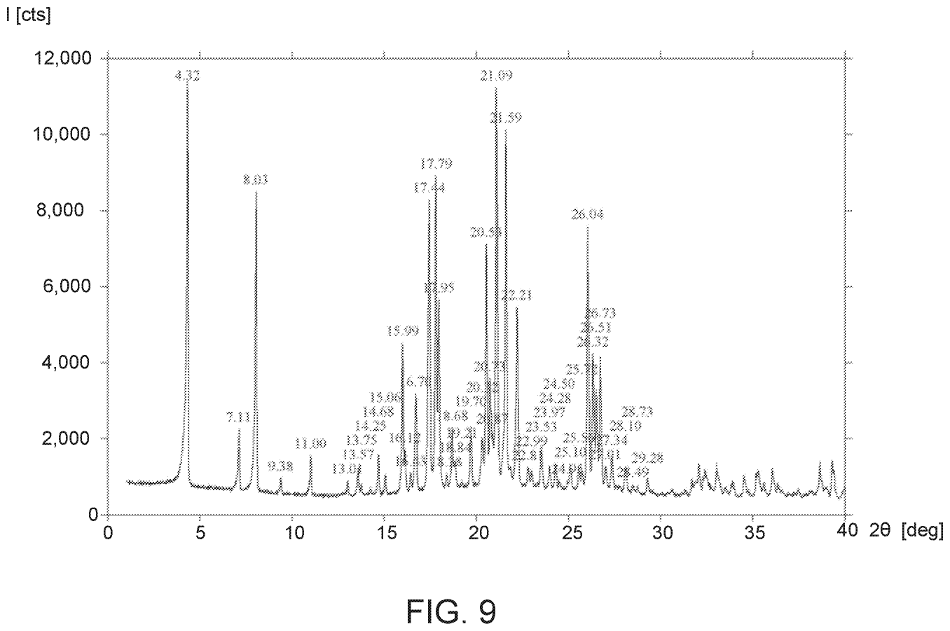

In one aspect, the crystalline form of Compound I is characterized by an XRPD pattern substantially as shown in FIG. 9.

In one aspect, the crystalline form of Compound I is characterized by the following XRPD pattern in Table 5 expressed in terms of the degree 2θ and relative intensities:

TABLE 5

| Angle | Relative |
|---|---|
| (Degree 2θ) | Intensity * (%) |
| 4.3 ± 0.2 | 100 |
| 7.1 ± 0.2 | 20 |
| 8 ± 0.2 | 71 |
| 9.4 ± 0.2 | 9 |
| 11 ± 0.2 | 14 |
| 13 ± 0.2 | 8 |
| 13.6 ± 0.2 | 11 |
| 13.8 ± 0.2 | 7 |
| 14.3 ± 0.2 | 6 |
| 14.7 ± 0.2 | 14 |
| 15.1 ± 0.2 | 9 |
| 16 ± 0.2 | 39 |
| 16.1 ± 0.2 | 15 |
| 16.4 ± 0.2 | 10 |
| 16.7 ± 0.2 | 28 |
| 17.4 ± 0.2 | 73 |
| 17.8 ± 0.2 | 77 |
| 18 ± 0.2 | 50 |
| 18.4 ± 0.2 | 9 |
| 18.7 ± 0.2 | 20 |
| 18.8 ± 0.2 | 13 |
| 19.2 ± 0.2 | 8 |
| 19.7 ± 0.2 | 20 |
| 20.3 ± 0.2 | 18 |
| 20.5 ± 0.2 | 63 |
| 20.7 ± 0.2 | 32 |
| 20.9 ± 0.2 | 19 |
| 21.1 ± 0.2 | 96 |
| 21.6 ± 0.2 | 88 |
| 22.2 ± 0.2 | 48 |
| 22.8 ± 0.2 | 11 |
| 23 ± 0.2 | 10 |
| 23.5 ± 0.2 | 15 |
| 24 ± 0.2 | 11 |
| 24.3 ± 0.2 | 12 |
| 24.5 ± 0.2 | 8 |
| 25 ± 0.2 | 8 |
| 25.1 ± 0.2 | 12 |
| 25.6 ± 0.2 | 11 |
| 25.7 ± 0.2 | 10 |
| 26 ± 0.2 | 67 |
| 26.3 ± 0.2 | 37 |
| 26.5 ± 0.2 | 29 |
| 26.7 ± 0.2 | 36 |
| 27 ± 0.2 | 11 |
| 27.3 ± 0.2 | 13 |
| 28.1 ± 0.2 | 10 |
| 28.5 ± 0.2 | 7 |
| 28.7 ± 0.2 | 7 |
| 29.3 ± 0.2 | 8 |

* The relative intensities can change depending on the crystal size and morphology.

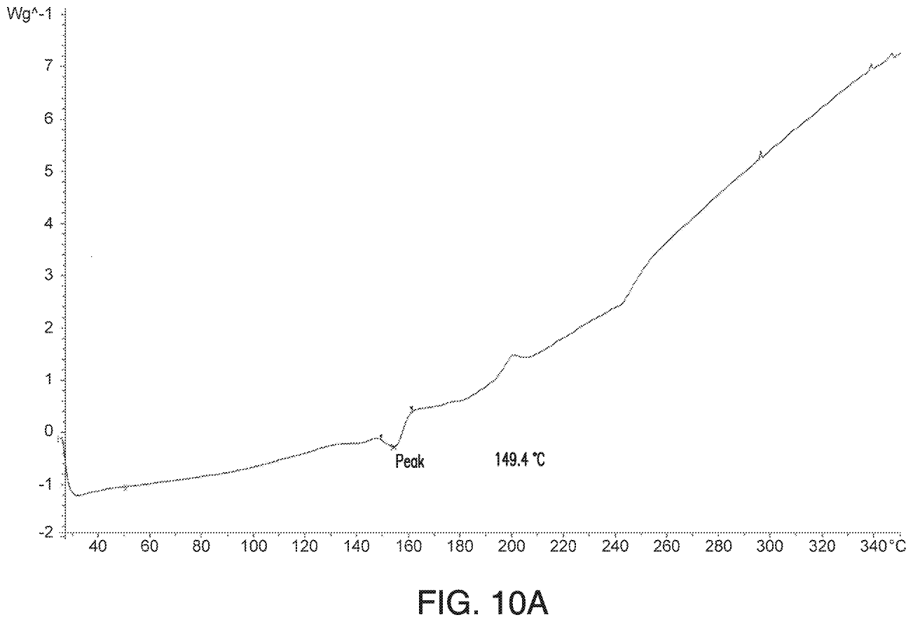

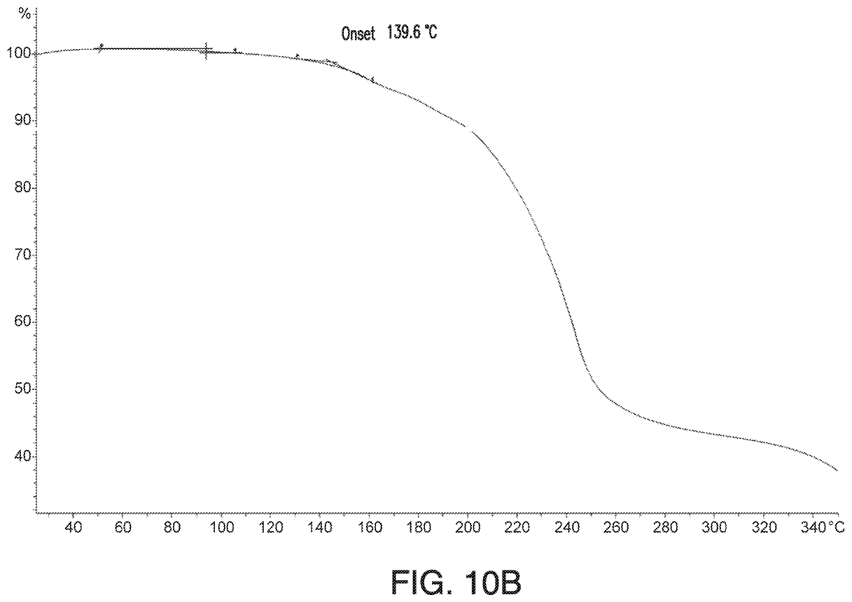

In one aspect, the crystalline form of Compound I is characterized by an endothermic peak with onset at about 149 °C, as determined by DSC. In one aspect, the crystalline form of Compound I is characterized by a DSC profile substantially as shown in FIG. 10A. In one aspect, the crystalline form of Compound I is characterized by a TGA profile substantially as shown in FIG. 10B.

In one aspect, the crystalline form is a solvate as determined by ¹H NMR. In one aspect, the crystalline form of Compound I is a dichloromethane solvate.

In one aspect, the crystalline form of Compound I has a unit cell that indexes as primitive monoclinic. In another aspect, the crystalline form of Compound I has a unit cell with an a value of about 5.541 Å, a b value of about 13.040 Å, and a c value of about 40.818 Å. In another aspect, the crystalline form of Compound I has a unit cell with a volume of about 2947.6 Å³.

In one aspect, the crystalline form of Compound I is Form C.

### D. Crystalline Form D

In one aspect, the present disclosure relates to a crystalline form of Compound I, characterized by an XRPD pattern having peaks at 5.3±0.2, 8.7±0.2, and 26.4±0.2 degrees two theta. In one aspect, the present disclosure relates to a crystalline form of Compound I, characterized by an XRPD pattern having peaks at 5.3±0.2, 8.7±0.2, 18.2±0.2, and 26.4±0.2 degrees two theta.

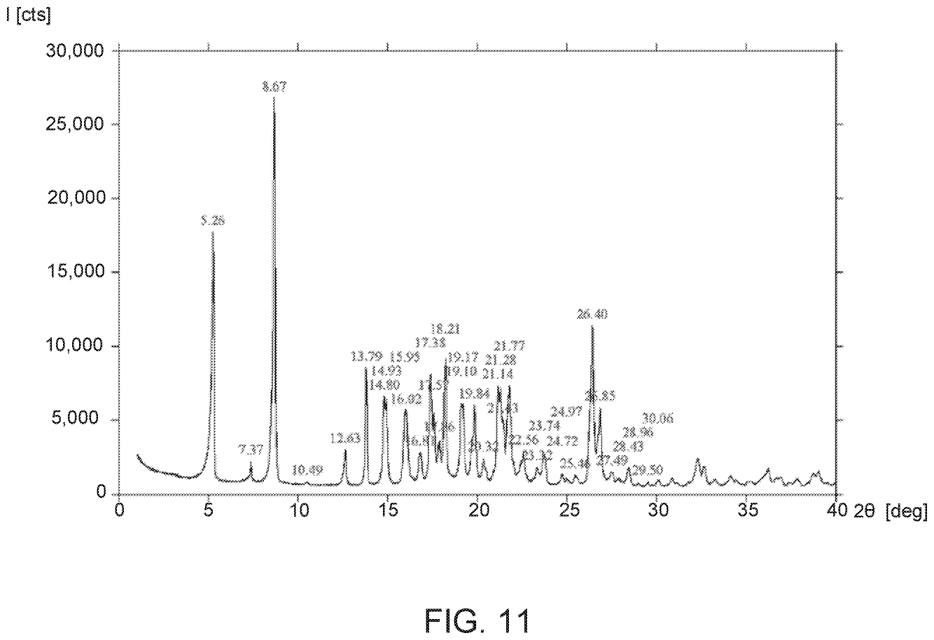

In one aspect, the crystalline form of Compound I is characterized by an XRPD pattern substantially as shown in FIG. 11.

In one aspect, the crystalline form of Compound I is characterized by the following XRPD pattern in Table 6 expressed in terms of the degree 2θ and relative intensities:

TABLE 6

| Angle | Relative |
|---|---|
| (Degree 2θ) | Intensity * (%) |
| 5.3 ± 0.2 | 66 |
| 7.4 ± 0.2 | 8 |
| 8.7 ± 0.2 | 100 |
| 10.5 ± 0.2 | 3 |
| 12.6 ± 0.2 | 11 |
| 13.8 ± 0.2 | 32 |
| 14.8 ± 0.2 | 25 |
| 14.9 ± 0.2 | 24 |
| 16.0 ± 0.2 | 22 |
| 16.0 ± 0.2 | 21 |
| 16.8 ± 0.2 | 11 |
| 17.4 ± 0.2 | 31 |
| 17.6 ± 0.2 | 21 |
| 17.9 ± 0.2 | 13 |
| 18.2 ± 0.2 | 34 |
| 19.1 ± 0.2 | 23 |
| 19.2 ± 0.2 | 23 |
| 19.8 ± 0.2 | 23 |
| 20.3 ± 0.2 | 9 |
| 21.1 ± 0.2 | 28 |
| 21.3 ± 0.2 | 27 |
| 21.4 ± 0.2 | 19 |
| 21.8 ± 0.2 | 28 |
| 22.6 ± 0.2 | 11 |
| 23.3 ± 0.2 | 7 |
| 23.7 ± 0.2 | 10 |
| 24.7 ± 0.2 | 5 |
| 25.0 ± 0.2 | 4 |
| 25.5 ± 0.2 | 5 |
| 26.4 ± 0.2 | 43 |
| 26.9 ± 0.2 | 22 |
| 27.5 ± 0.2 | 6 |
| 28.4 ± 0.2 | 7 |
| 29.0 ± 0.2 | 3 |
| 29.5 ± 0.2 | 3 |
| 30.1 ± 0.2 | 4 |

* The relative intensities can change depending on the crystal size and morphology.

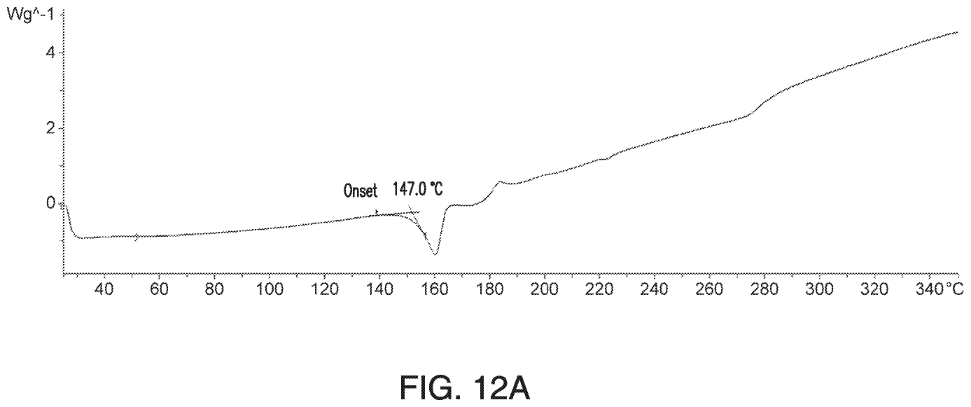

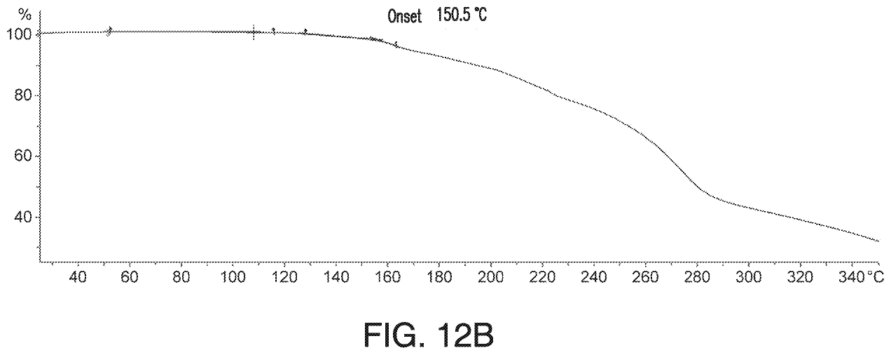

In one aspect, the crystalline form of Compound I is characterized by an endothermic peak with onset at about 147 °C, as determined by DSC. In one aspect, the crystalline form of Compound I is characterized by a DSC profile substantially as shown in FIG. 12A. In one aspect, the crystalline form of Compound I is characterized by a TGA profile substantially as shown in FIG. 12B.

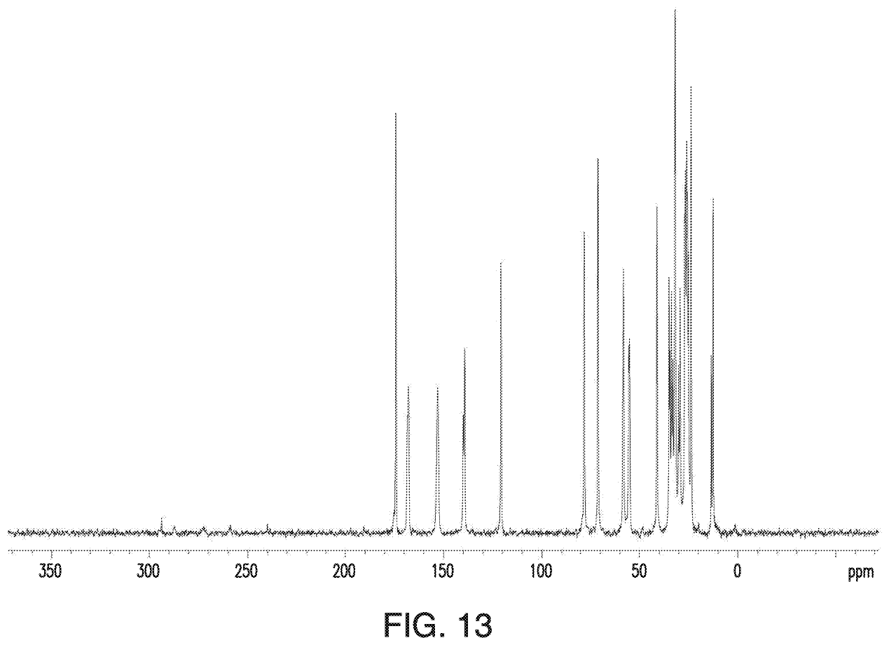

In one aspect, the crystalline form of Compound I is characterized by a ¹³C solid state NMR substantially similar to FIG. 13.

In one aspect, the crystalline form of Compound I is Form D.

### E. Crystalline Form E

In one aspect, the present disclosure relates to a crystalline form of Compound I, characterized by an XRPD pattern having peaks at 5.8±0.2, 17.9±0.2, and 18.9±0.2 degrees two theta. In one aspect, the present disclosure relates to a crystalline form of Compound I, characterized by an XRPD pattern having peaks at 5.8±0.2, 17.9±0.2, 18.9±0.2, and 20.7±0.2 degrees two theta.

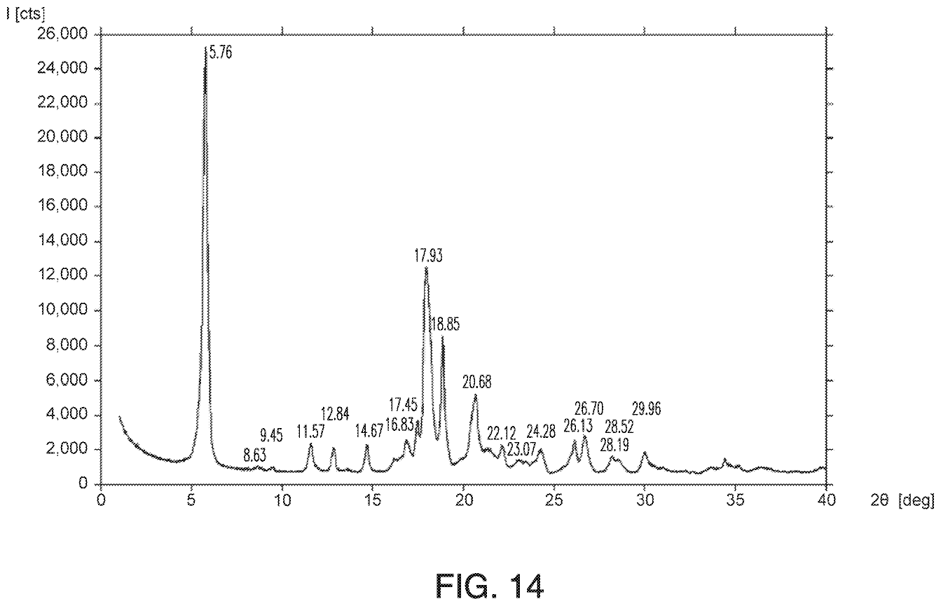

In one aspect, the crystalline form of Compound I is characterized by an XRPD pattern substantially as shown in FIG. 14.

In one aspect, the crystalline form of Compound I is characterized by the following XRPD pattern in Table 7 expressed in terms of the degree 2θ and relative intensities:

TABLE 7

| Angle | Relative |
|---|---|
| (Degree 2θ) | Intensity * (%) |
| 5.8 ± 0.2 | 100 |
| 8.6 ± 0.2 | 4 |
| 9.5 ± 0.2 | 4 |
| 11.6 ± 0.2 | 9 |
| 12.8 ± 0.2 | 8 |
| 14.7 ± 0.2 | 9 |
| 16.9 ± 0.2 | 10 |
| 17.5 ± 0.2 | 14 |
| 17.9 ± 0.2 | 49 |
| 18.9 ± 0.2 | 34 |
| 20.7 ± 0.2 | 20 |
| 22.1 ± 0.2 | 9 |
| 23.1 ± 0.2 | 5 |
| 24.3 ± 0.2 | 8 |
| 26.1 ± 0.2 | 10 |
| 26.7 ± 0.2 | 11 |
| 28.2 ± 0.2 | 6 |
| 28.5 ± 0.2 | 6 |
| 30.0 ± 0.2 | 7 |

* The relative intensities can change depending on the crystal size and morphology.

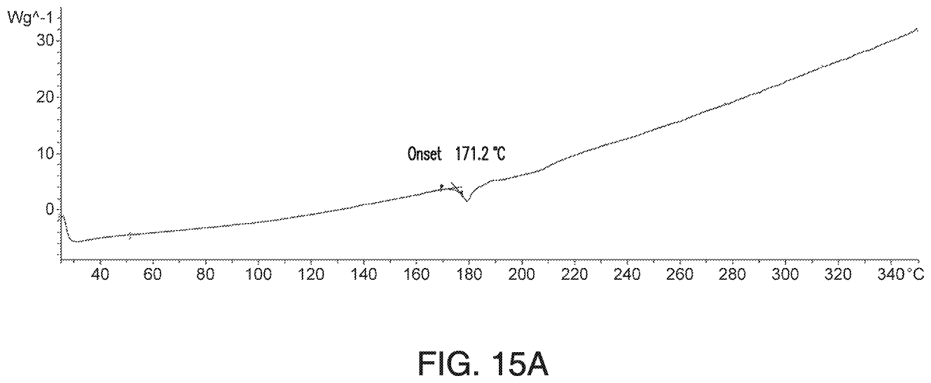

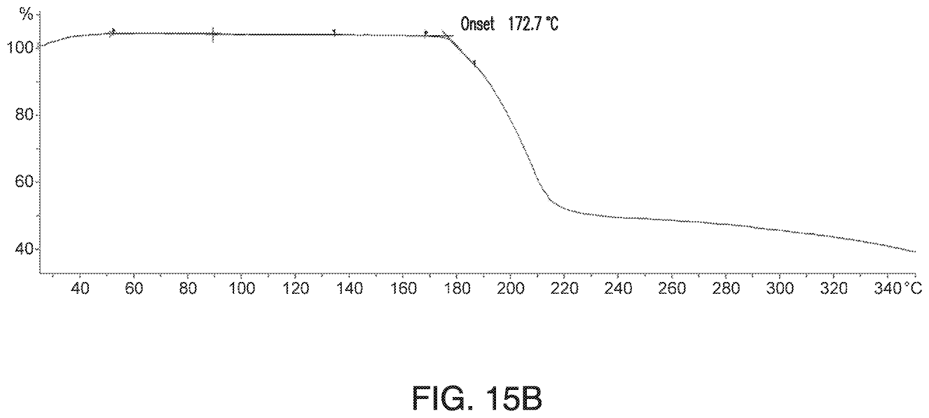

In one aspect, the crystalline form of Compound I is characterized by an endothermic peak with onset at about 171 °C, as determined by DSC. In one aspect, the crystalline form of Compound I is characterized by a DSC profile substantially as shown in FIG. 15A. In one aspect, the crystalline form of Compound I is characterized by a TGA profile substantially as shown in FIG. 15B.

In one aspect, the crystalline form of Compound I is Form E.

### F. Crystalline Form F

In one aspect, the present disclosure relates to a crystalline form of Compound I, characterized by an XRPD pattern having peaks at 3.8±0.2, 9.5±0.2, and 16.8±0.2 degrees two theta. In one aspect, the present disclosure relates to a crystalline form of Compound I, characterized by an XRPD pattern having peaks at 3.8±0.2, 9.5±0.2, 16.8±0.2, and 17.9±0.2 degrees two theta.

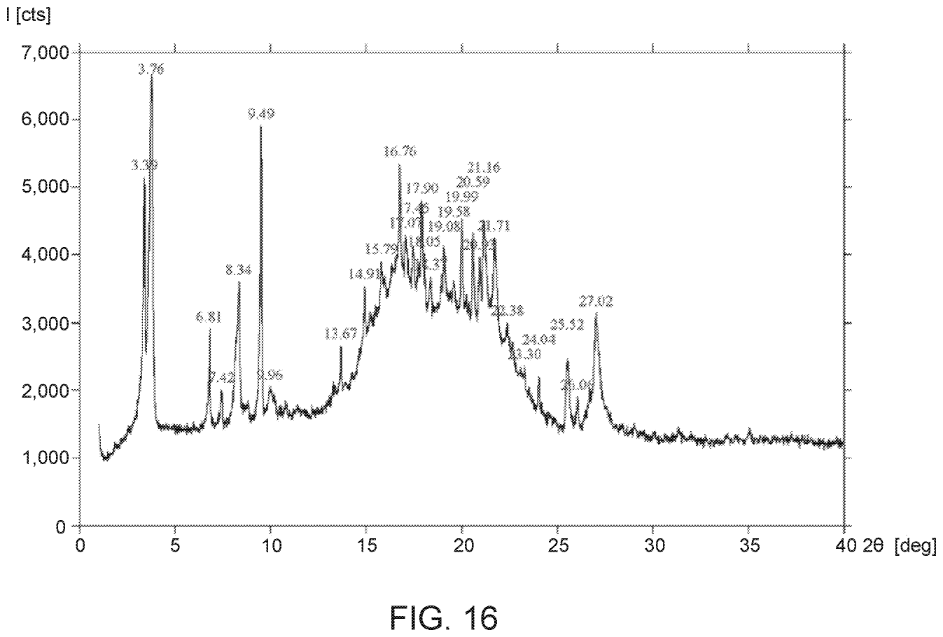

In one aspect, the crystalline form of Compound I is characterized by an XRPD pattern substantially as shown in FIG. 16.

In one aspect, the crystalline form of Compound I is characterized by the following XRPD pattern in Table 8 expressed in terms of the degree 2θ and relative intensities:

TABLE 8

| Angle | Relative |
|---|---|
| (Degree 2θ) | Intensity * (%) |
| 3.4 ± 0.2 | 76 |
| 3.8 ± 0.2 | 100 |
| 6.8 ± 0.2 | 43 |
| 7.4 ± 0.2 | 30 |
| 8.3 ± 0.2 | 53 |
| 9.5 ± 0.2 | 89 |
| 10.0 ± 0.2 | 31 |
| 13.7 ± 0.2 | 40 |
| 14.9 ± 0.2 | 53 |
| 15.8 ± 0.2 | 59 |
| 16.8 ± 0.2 | 80 |
| 17.1 ± 0.2 | 63 |
| 17.5 ± 0.2 | 62 |
| 17.9 ± 0.2 | 72 |
| 18.1 ± 0.2 | 60 |
| 18.4 ± 0.2 | 54 |
| 19.1 ± 0.2 | 60 |
| 19.6 ± 0.2 | 54 |
| 20.0 ± 0.2 | 67 |
| 20.6 ± 0.2 | 65 |
| 20.9 ± 0.2 | 58 |
| 21.2 ± 0.2 | 67 |
| 21.7 ± 0.2 | 64 |
| 22.4 ± 0.2 | 45 |
| 23.3 ± 0.2 | 34 |
| 24.0 ± 0.2 | 32 |
| 25.5 ± 0.2 | 37 |
| 26.1 ± 0.2 | 29 |
| 27.0 ± 0.2 | 47 |

* The relative intensities can change depending on the crystal size and morphology.

In one aspect, the crystalline form of Compound I is Form F.

### G. Crystalline Form G

In one aspect, the present disclosure relates to a crystalline form of Compound I, characterized by an XRPD pattern having peaks at 3.4±0.2, 21.2±0.2, and 21.9±0.2 degrees two theta. In one aspect, the present disclosure relates to a crystalline form of Compound I, characterized by an XRPD pattern having peaks at 3.4±0.2, 21.2±0.2, 21.9±0.2, and 22.4±0.2 degrees two theta.

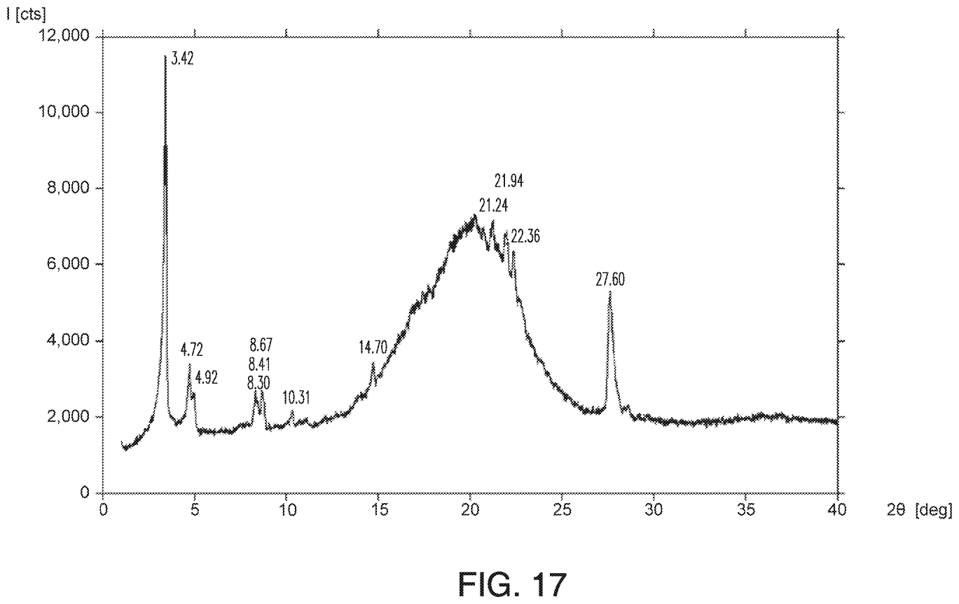

In one aspect, the crystalline form of Compound I is characterized by an XRPD pattern substantially as shown in FIG. 17.

In one aspect, the crystalline form of Compound I is characterized by the following XRPD pattern in Table 9 expressed in terms of the degree 2θ and relative intensities:

TABLE 9

| Angle | Relative |
|---|---|
| (Degree 2θ) | Intensity * (%) |
| 3.4 ± 0.2 | 100 |
| 4.7 ± 0.2 | 30 |
| 4.9 ± 0.2 | 23 |
| 8.3 ± 0.2 | 23 |
| 8.4 ± 0.2 | 22 |
| 8.7 ± 0.2 | 24 |
| 10.3 ± 0.2 | 19 |
| 14.7 ± 0.2 | 30 |
| 21.2 ± 0.2 | 62 |
| 21.9 ± 0.2 | 59 |
| 22.4 ± 0.2 | 55 |
| 27.6 ± 0.2 | 45 |

* The relative intensities can change depending on the crystal size and morphology.

In one aspect, the crystalline form of Compound I is Form G.

### H. Crystalline Form H

In one aspect, the present disclosure relates to a crystalline form of Compound I, characterized by an XRPD pattern having peaks at 3.8±0.2, 5.3±0.2, and 8.5±0.2 degrees two theta. In one aspect, the present disclosure relates to a crystalline form of Compound I, characterized by an XRPD pattern having peaks at 3.8±0.2, 5.3±0.2, 8.5±0.2, and 15.9±0.2 degrees two theta.

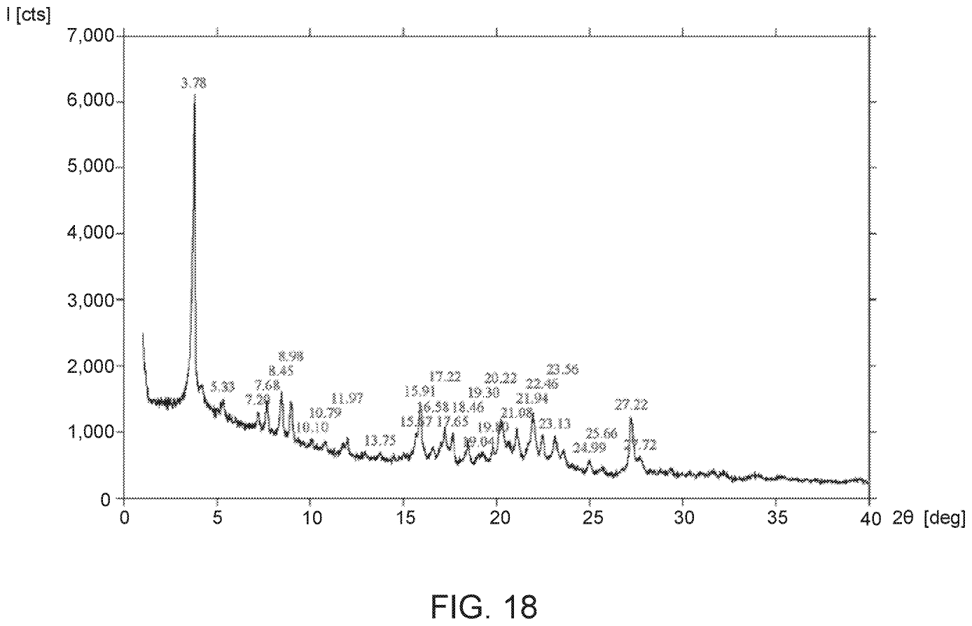

In one aspect, the crystalline form of Compound I is characterized by an XRPD pattern substantially as shown in FIG. 18.

In one aspect, the crystalline form of Compound I is characterized by the following XRPD pattern in Table 10 expressed in terms of the degree 2θ and relative intensities:

TABLE 10

| Angle | Relative |
|---|---|
| (Degree 2θ) | Intensity * (%) |
| 3.8 ± 0.2 | 100 |
| 5.3 ± 0.2 | 24 |
| 7.2 ± 0.2 | 21 |
| 7.7 ± 0.2 | 23 |
| 8.5 ± 0.2 | 26 |
| 9.0 ± 0.2 | 24 |
| 10.1 ± 0.2 | 15 |
| 10.8 ± 0.2 | 14 |
| 12.0 ± 0.2 | 14 |
| 13.8 ± 0.2 | 11 |
| 15.7 ± 0.2 | 16 |
| 15.9 ± 0.2 | 24 |
| 16.6 ± 0.2 | 13 |
| 17.2 ± 0.2 | 17 |
| 17.7 ± 0.2 | 16 |
| 18.5 ± 0.2 | 14 |
| 19.0 ± 0.2 | 11 |
| 19.3 ± 0.2 | 11 |
| 19.8 ± 0.2 | 12 |
| 20.2 ± 0.2 | 18 |
| 21.1 ± 0.2 | 16 |
| 21.9 ± 0.2 | 20 |
| 22.5 ± 0.2 | 15 |
| 23.1 ± 0.2 | 15 |
| 23.6 ± 0.2 | 12 |
| 25.0 ± 0.2 | 9 |
| 25.7 ± 0.2 | 8 |
| 27.2 ± 0.2 | 19 |
| 27.7 ± 0.2 | 10 |

* The relative intensities can change depending on the crystal size and morphology.

In one aspect, the crystalline form of Compound I is Form H.

### I. Crystalline Form I

In one aspect, the present disclosure relates to a crystalline form of Compound I, characterized by an XRPD pattern having peaks at 5.0±0.2, 16.8±0.2, and 18.8±0.2 degrees two theta. In one aspect, the present disclosure relates to a crystalline form of Compound I, characterized by an XRPD pattern having peaks at 5.0±0.2, 15.9±0.2, 16.8±0.2, and 18.8±0.2 degrees two theta.

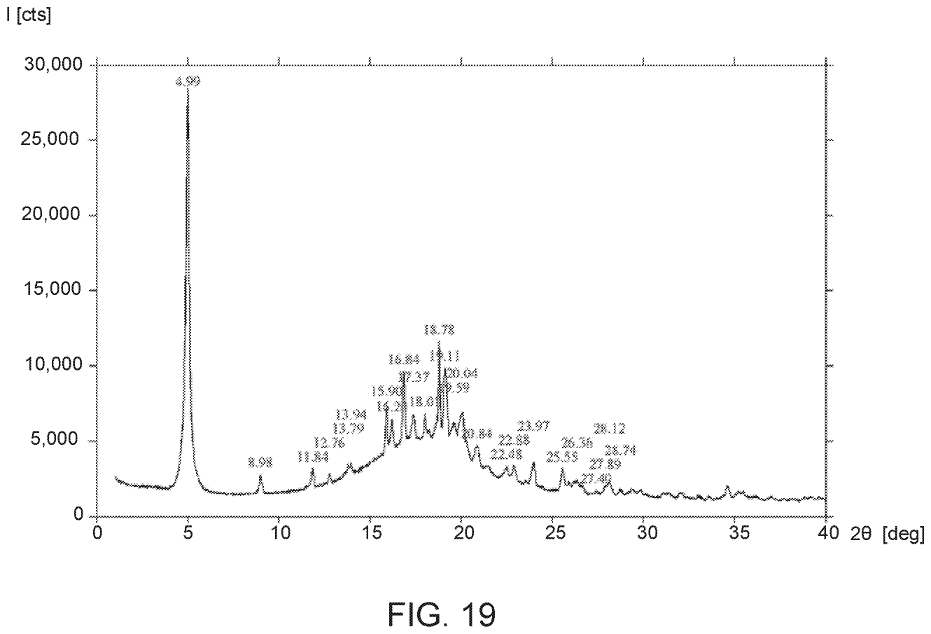

In one aspect, the crystalline form of Compound I is characterized by an XRPD pattern substantially as shown in FIG. 19.

In one aspect, the crystalline form of Compound I is characterized by the following XRPD pattern in Table 11 expressed in terms of the degree 2θ and relative intensities:

TABLE 11

| Angle | Relative |
|---|---|
| (Degree 2θ) | Intensity * (%) |
| 5.0 ± 0.2 | 100 |
| 9.0 ± 0.2 | 10 |
| 11.8 ± 0.2 | 11 |
| 12.8 ± 0.2 | 10 |
| 13.8 ± 0.2 | 12 |
| 13.9 ± 0.2 | 12 |
| 15.9 ± 0.2 | 27 |
| 16.2 ± 0.2 | 23 |
| 16.8 ± 0.2 | 34 |
| 17.4 ± 0.2 | 24 |
| 18.0 ± 0.2 | 24 |
| 18.8 ± 0.2 | 41 |
| 19.1 ± 0.2 | 35 |
| 19.6 ± 0.2 | 22 |
| 20.0 ± 0.2 | 24 |
| 20.8 ± 0.2 | 16 |
| 22.5 ± 0.2 | 12 |
| 22.9 ± 0.2 | 12 |
| 24.0 ± 0.2 | 12 |
| 25.6 ± 0.2 | 11 |
| 26.4 ± 0.2 | 9 |
| 27.4 ± 0.2 | 6 |
| 27.9 ± 0.2 | 7 |
| 28.1 ± 0.2 | 8 |
| 28.7 ± 0.2 | 6 |

* The relative intensities can change depending on the crystal size and morphology.

In one aspect, the crystalline form of Compound I is Form I.

### J. Crystalline Form J

In one aspect, the present disclosure relates to a crystalline form of Compound I, characterized by an XRPD pattern having peaks at 5.9±0.2, 17.4±0.2, and 18.8±0.2 degrees two theta. In one aspect, the present disclosure relates to a crystalline form of Compound I, characterized by an XRPD pattern having peaks at 5.9±0.2, 12.7±0.2, 17.4±0.2, and 18.8±0.2 degrees two theta.

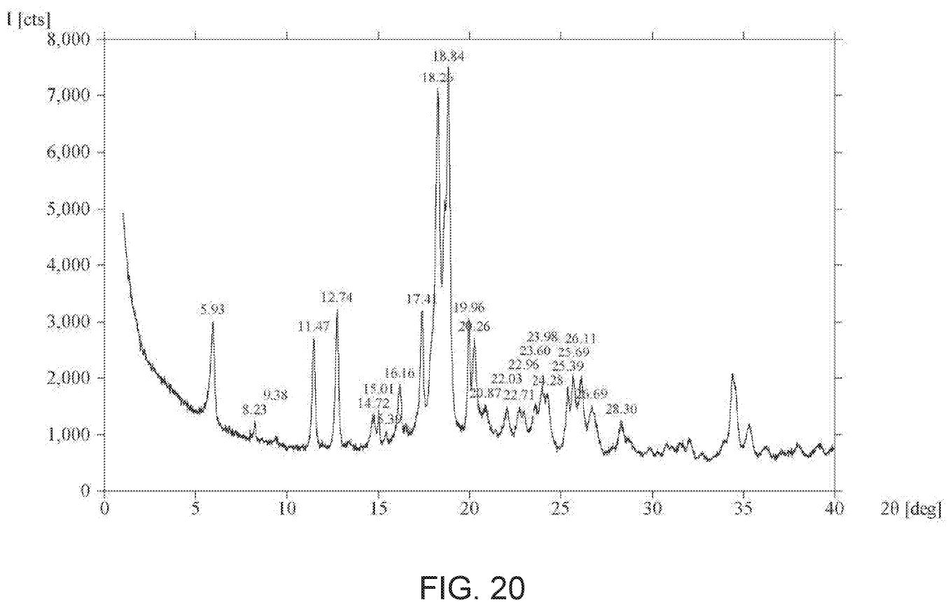

In one aspect, the crystalline form of Compound I is characterized by an XRPD pattern substantially as shown in FIG. 20.

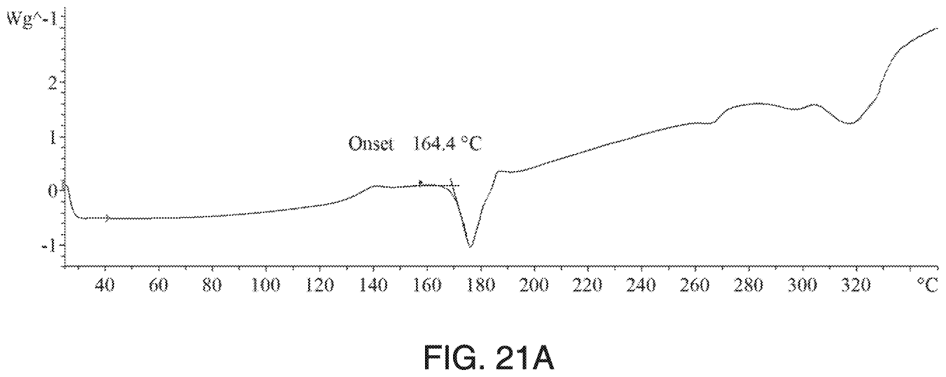

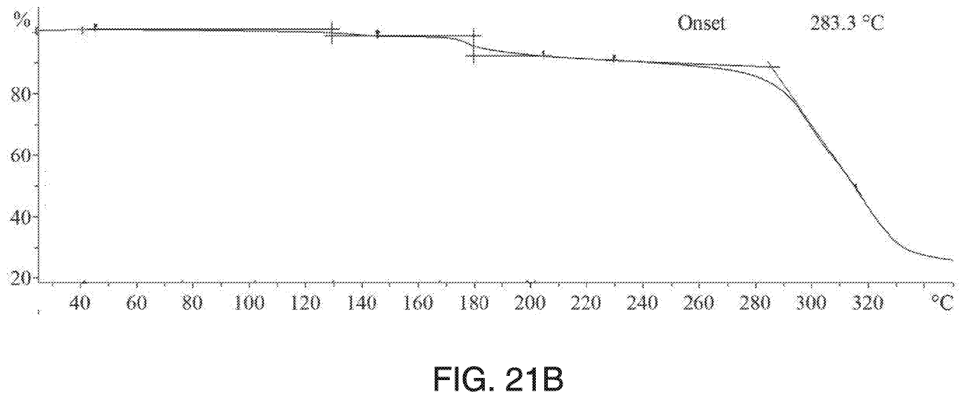

In one aspect, the crystalline form of Compound I is characterized by an endothermic peak with onset at about 164 °C, as determined by DSC. In one aspect, the crystalline form of Compound I is characterized by a DSC profile substantially as shown in FIG. 21A. In one aspect, the crystalline form of Compound I is characterized by a TGA profile substantially as shown in FIG. 21B.

In one aspect, the crystalline form of Compound I is characterized by the following XRPD pattern in Table 12 expressed in terms of the degree 2θ and relative intensities:

TABLE 12

| Angle | Relative |
|---|---|
| (Degree 2θ) | Intensity * (%) |
| 5.9 ± 0.20 | 40 |
| 8.2 ± 0.20 | 16 |
| 9.4 ± 0.20 | 13 |
| 11.5 ± 0.20 | 36 |
| 12.7 ± 0.20 | 42 |
| 14.7 ± 0.20 | 17 |
| 15.0 ± 0.20 | 19 |
| 15.4 ± 0.20 | 14 |
| 16.2 ± 0.20 | 25 |
| 17.4 ± 0.20 | 43 |
| 18.3 ± 0.20 | 93 |
| 18.8 ± 0.20 | 100 |
| 20.0 ± 0.20 | 41 |
| 20.3 ± 0.20 | 36 |
| 20.9 ± 0.20 | 20 |
| 22.0 ± 0.20 | 20 |
| 22.7 ± 0.20 | 20 |
| 23.0 ± 0.20 | 18 |
| 23.6 ± 0.20 | 20 |
| 24.0 ± 0.20 | 24 |
| 24.3 ± 0.20 | 22 |
| 25.4 ± 0.20 | 24 |
| 25.7 ± 0.20 | 27 |
| 26.1 ± 0.20 | 26 |
| 26.7 ± 0.20 | 20 |

* The relative intensities can change depending on the crystal size and morphology.

In one aspect, the crystalline form of Compound I is Form J.

In some aspects, any one of the crystalline forms discussed above is substantially free of other polymorphic forms. In some aspects, the crystalline form has a polymorphic purity of at least about 80%. In some aspects, the crystalline form has a polymorphic purity of at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99%.

In one aspect, the crystalline form of Compound I is selected from the group consisting of Form A, Form B, Form C, Form D, Form E, Form F, Form G, Form H, Form I, and Form J. In one aspect, the crystalline form of Compound I is Form A.

In one aspect, the crystalline form of Compound I is a mixture of two or more forms selected from the group consisting of Form A, Form B, Form C, Form D, Form E, Form F, Form G, Form H, Form I, and Form J. In another aspect, the crystalline form of Compound I is a mixture of two or more forms selected from the group consisting of Form A, Form B, and Form C. In another aspect, the crystalline form of Compound I is a mixture of Form A and Form B, where Form B is the major form and Form A is the minor form.

In some aspects, the present disclosure provides a methods of making a crystalline form of Compound I where the crystalline form is selected from the group consisting of Form A, Form B, Form C, Form D, Form E, Form F, Form G, Form H, Form I, and Form J. One or more methods to prepare Forms A-J are provided in Experimental Section herein.

### III. Pharmaceutical Composition

The present disclosure relates to a pharmaceutical composition comprising the crystalline form of any one of Forms A-J of Compound I and a pharmaceutically acceptable carrier, diluent, or excipients, or a mixture thereof.

In one aspect, the pharmaceutical composition comprises the crystalline form of any one of Forms A-J of Compound I.

Pharmaceutical compositions comprising the crystalline form of any one of Forms A-J of Compound I can be in a form suitable for oral use, for example, as tablets, troches, lozenges, dispersible powders or granules, or hard or soft capsules. Compositions intended for oral use can be prepared according to any known method, and such compositions can contain one or more agents selected from the group consisting of sweetening agents, flavoring agents, coloring agents, and preserving agents in order to provide pharmaceutically elegant and palatable preparations.

In some aspects, the pharmaceutical composition can be administered to subjects via the oral, parenteral (such as subcutaneous, intravenous, intramuscular, intrasternal and infusion techniques), rectal, intranasal, topical or transdermal (e.g., through the use of a patch) routes.

In one aspect, the pharmaceutical composition comprises about 100 mg to about 1500 mg, about 100 mg to about 1400 mg, about 100 mg to about 1300 mg, about 100 mg to about 1200 mg, about 100 mg to about 1100 mg, about 100 mg to about 1000 mg, about 100 mg to about 900 mg, about 100 mg to about 800 mg, about 100 mg to about 700 mg, about 100 mg to about 600 mg, about 100 mg to about 500 mg, about 100 mg to about 400 mg, about 100 mg to about 300 mg, about 100 mg to about 200 mg, or about 100 mg to about 150 mg of the crystalline form of any one of Forms A-J of Compound I disclosed herein. In one aspect, the pharmaceutical composition comprises about 100 mg, about 200 mg, about 300 mg, about 400 mg, about 500 mg, about 600 mg, about 700 mg, about 800 mg, about 900 mg, about 1000 mg, about 1100 mg, about 1200 mg, about 1300 mg, about 1400 mg, or about 1500 mg of the crystalline form of any one of Forms A-J of Compound I disclosed herein.

In some aspects, the pharmaceutical composition is an oral tablet. In some aspects, the oral tablet comprises about 0.1 mg to 2000 mg of the crystalline form of any one of Forms A-J 2-[3-cyclohexyl-3-(trans-4-propoxy-cyclohexyl)-ureido]-thiazol-5-ylsulfanyl}-acetic acid. In some aspects, the oral tablet comprises about 1 mg to about 2000 mg of the crystalline form of any one of Forms A-J of Compound I. In some aspects, the oral tablet comprises about 1 mg to about 1000 mg of the crystalline form of any one of Forms A-J of Compound I. In some aspects, the oral tablet comprises about 100 mg to about 800 mg of the crystalline form of any one of Forms A-J of Compound I. In some aspects, the oral tablet comprises about 50 mg to about 400 mg of the crystalline form of any one of Forms A-J of Compound I. In some aspects, the oral tablet comprises about 100 mg to about 400 mg of the crystalline form of any one of Forms A-J of Compound I. In some aspects, the oral tablet comprises about 100 mg to about 300 mg of the crystalline form of any one of Forms A-J of Compound I. In some aspects, the oral tablet comprises about 500 mg to about 1000 mg of the crystalline form of any one of Forms A-J of Compound I. In some aspects, the oral tablet comprises about 0.1 mg, about 0.5 mg, about 1 mg, about 5 mg, about 10 mg, about 20 mg, about 30 mg, about 40 mg, about 50 mg, about 60 mg, about 70 mg, about 80 mg, about 90 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 550 mg, about 600 mg, about 650 mg, about 700 mg, about 750 mg, about 800 mg, about 850 mg, about 900 mg, about 1000 mg, about 1050 mg, about 1100 mg, about 1150 mg, about 1200 mg, about 1250 mg, about 1300 mg, about 1350 mg, about 1400 mg, about 1450 mg, about 1500 mg, about 1550 mg, about 1600 mg, about 1650 mg, about 1700 mg, about 1750 mg, about 1800 mg, about 1850 mg, about 1900 mg, about 1950 mg, or about 2000 mg of the crystalline form of any one of Forms A-J of Compound I. In some aspects, the oral tablet comprises 800 mg of the crystalline form of any one of Forms A-J of Compound I. In some aspects, the oral tablet comprises 400 mg of the crystalline form of any one of Forms A-J of Compound I. In some aspects, the oral tablet comprises 300 mg of the crystalline form of any one of Forms A-J of Compound I. In some aspects, the oral tablet comprises about 200 mg of the crystalline form of any one of Forms A-J of Compound I.

### IV. Method of Treatment

In some aspects, the present disclosure relates to a method of treating a type of diabetes mellitus, wherein the method comprises administering the pharmaceutical composition discussed above to a patient in need thereof. The method can comprise administering a pharmaceutical composition comprising a therapeutically effective amount of the crystalline form of any one of Forms A-J of Compound I. In some aspects, the type of diabetes mellitus is type 1 diabetes. In some aspects, the type of diabetes mellitus is type 2 diabetes. In some aspects, the type of diabetes is one or two types of type 1 diabetes and type 2 diabetes.

In some aspects, the patient is being treated with an insulin therapy. In some aspects, the insulin therapy is a continuous insulin infusion. In some aspects, the insulin therapy is a continuous subcutaneous insulin infusion. In some aspects, the insulin therapy is a multiple daily doses of insulin.

In another aspect, the present disclosure provides a method for the treatment of glucokinase-deficiency mediated conditions or diseases, or conditions benefiting from an increase in glucokinase activity, comprising administering to a subject in need thereof a compound or a pharmaceutical composition of the present disclosure.

In another aspect, the present disclosure provides a method for treatment of metabolic disorders, for blood glucose lowering, for the treatment of hyperglycemia, for the treatment of hypoglycemia for the treatment of impaired glucose tolerance (IGT), for the treatment of Syndrome X, for the treatment of impaired fasting glucose (IFG), for delaying the progression of impaired glucose tolerance (IGT) to type 2 diabetes, for delaying the progression of non-insulin requiring type 2 diabetes to insulin requiring type 2 diabetes, for the treatment of dyslipidemia, for the treatment of hyperlipidemia, for the treatment of hypertension, for lowering of food intake, for appetite regulation, for the treatment of obesity, for regulating feeding behavior, or for enhancing the secretion of enteroincretins, comprising administering to a subject in need of such treatment a compound or a pharmaceutical composition of the present disclosure.

In another aspect, the present disclosure provides a method for the preservation of beta-cell mass and function comprising administering to a subject in need of such treatment a compound or a pharmaceutical composition of the present disclosure.

In another aspect, the present disclosure provides a method of preserving and/or increasing beta-cell mass and function in a subject having undergone pancreatic islet transplantation comprising administering to a subject in need of such treatment a compound or a pharmaceutical composition of the present disclosure.

In another aspect, the present disclosure provides a method of improving liver function and/or survival in subjects undergoing liver transplantation comprising administering to a subject in need of such treatment a compound or a pharmaceutical composition of the present disclosure. In a further aspect, the administration occurs before, during or after transplantation, or any combination thereof.

In another aspect, the present disclosure provides a method of preventing diabetic ketoacidosis or reducing the occurrence of diabetic ketoacidosis events in a subject comprising administering to a subject in need of such treatment a compound or a pharmaceutical composition of the present disclosure.

Depending on the condition, disorder, or disease to be treated and the subject's condition, the pharmaceutical compositions provided herein can be administered by oral, parenteral (e.g., intramuscular, intraperitoneal, intravenous or intraarterial (e.g., via catheter), ICV, intracistemal injection or infusion, subcutaneous injection, or implant), inhalation, nasal, vaginal, rectal, sublingual, and/or topical (e.g., transdermal or local) routes of administration, and can be formulated alone or together in suitable dosage unit with a pharmaceutically acceptable vehicle, carrier, diluent, excipient, or a mixture thereof, appropriate for each route of administration. In one aspect, the pharmaceutical composition is administered orally.

For oral administration, the pharmaceutical compositions provided herein can be provided in solid, semisolid, or liquid dosage forms for oral administration. As used herein, oral administration also includes buccal, lingual, and sublingual administration. Suitable oral dosage forms include, but are not limited to, tablets, capsules, pills, troches, lozenges, pastilles, cachets, pellets, medicated chewing gum, granules, bulk powders, effervescent or non-effervescent powders or granules, solutions, emulsions, suspensions (e.g., aqueous or oil suspensions), wafers, sprinkles, elixirs, syrups, bolus, electuaries, or pastes. In one aspect, the pharmaceutical composition is administered as a tablet.

The dose can be in the form of one, two, three, four, five, six, or more sub-doses that are administered at appropriate intervals per day. The dose or sub-doses can be administered in the form of dosage units containing from about 1 mg to about 2000 mg, from about 10 mg to about 2000 mg, from about 100 mg to about 1500 mg, from about 200 mg to about 1500 mg, from about 200 mg to about 1500 mg, from about 300 mg to about 1500 mg, from about 400 mg to about 1500 mg, from about 500 mg to about 1500 mg, from about 500 mg to about 1000 mg, or from about 500 mg to about 800 mg of the crystalline form of any one of Forms A-J per dosage unit. For example, the dose or subdoses can be administered in the form of dosage units containing about 100 mg, about 200 mg, about 300 mg, about 400 mg, about 500 mg, about 600 mg, about 700 mg, about 800 mg, about 900 mg, about 1000 mg, about 1100 mg, about 1200 mg, about 1300 mg, about 1400 mg, about 1500 mg, about 1600 mg, about 1700 mg, about 1800 mg, about 1900 mg, or about 2000 mg of the crystalline form of any one of Forms A-J disclosed herein.

In some aspects, the patient is administered about 0.1 mg to about 2000 mg of the crystalline form of any one of Forms A-J of Compound I daily. In some aspects, the patient is administered about 1 mg to about 2000 mg of the crystalline form of any one of Forms A-J of Compound I daily. In some aspects, the patient is administered about 100 mg to about 800 mg of the crystalline form of any one of Forms A-J of Compound I daily. In some aspects, the patient is administered about 50 mg to about 400 mg of the crystalline form of any one of Forms A-J of Compound I daily. In some aspects, the patient is administered about 100 mg to about 400 mg of the crystalline form of any one of Forms A-J of Compound I daily. In some aspects, the patient is administered about 100 mg to about 300 mg of the crystalline form of any one of Forms A-J of Compound I daily. In some aspects, the patient is administered about 500 mg to about 1000 mg of the crystalline form of any one of Forms A-J of Compound I daily. In some aspects, the patient is administered about 0.1 mg, about 0.5 mg, about 1 mg, about 5 mg, about 10 mg, about 20 mg, about 30 mg, about 40 mg, about 50 mg, about 60 mg, about 70 mg, about 80 mg, about 90 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 500 mg, about 550 mg, about 1000 mg, about 1050 mg, about 1100 mg, about 1150 mg, about 1200 mg, about 1250 mg, about 1300 mg, about 1350 mg, about 1400 mg, about 1450 mg, about 1500 mg, about 1550 mg, about 1600 mg, about 1650 mg, about 1700 mg, about 1750 mg, about 1800 mg, about 1850 mg, about 1900 mg, about 1950 mg, or about 2000 mg, of the crystalline form of any one of Forms A-J of Compound I once daily. In some aspects, the patient is administered about 800 mg of the crystalline form of any one of Forms A-J of Compound I once daily. In some aspects, the patient is administered about 400 mg of the crystalline form of any one of Forms A-J of Compound I once daily. In some aspects, the patient is administered about 300 mg of the crystalline form of any one of Forms A-J of Compound I once daily. In some aspects, the patient is administered about 200 mg of the crystalline form of any one of Forms A-J of Compound I once daily. In some aspects, the patient is administered about 100 mg of the crystalline form of any one of Forms A-J of Compound I once daily.

### EXAMPLES

### A. Abbreviations and Acronyms

| | |
|---|---|
| DSC | Differential scanning calorimetry |
| DVS | Dynamic vapor sorption |
| HSM | Hot-stage microscopy |
| NMR | Nuclear magnetic resonance spectroscopy |
| PLM | Polarized light microscopy |
| TGA | Thermogravimetric analysis |
| XRPD | X-ray powder diffraction |
| CC | Crash cool |
| FC | Fast cool |
| FE | Fast evaporation |
| SAS | Solvent/antisolvent |
| SE | Slow evaporation |
| ACN | Acetonitrile |
| 1-BuOH | 1-Butanol |
| 2-BuOH | 2-Butanol |
| BuOAc | Butyl acetate |
| iBuOAc | Isobutyl acetate |
| t-BuOAc | tert-Butyl acetate |
| CHCl₃ | Chloroform |
| DCE | 1,2-Dichloroethane |
| DCM | Dichloromethane |
| DMF | Dimethylformamide |
| DMSO | Dimethyl sulfoxide |
| EtOAc | Ethyl acetate |
| EtOH | Ethanol |
| HFIPA | Hexafluoroisopropanol or |
| | Hexafluoro-2-propanol |
| H2O | Water |
| IPA | Isopropyl alcohol or 2-propanol |
| MEK | Methyl ethyl ketone or butanone |
| MeOH | Methanol |
| MIBK | Methyl isobutyl ketone |
| MTBE | Methyl tert-butyl ether |
| NMP | N-Methyl-2-Pyrrolidone |
| iPrOAc | Isopropyl acetate |
| 1-PrOH | 1-Propanol |
| TFE | 2,2,2-Trifluoroethanol |
| THF | Tetrahydrofuran |
| agg. | Aggregates |
| anh. | Anhydrous |
| B/E | Birefringence and extinction |
| d | Day(s) |
| endo | Endotherm |
| h | hour(s) |
| min | minute(s) |
| NA | numerical aperture |
| RH | Relative humidity |
| RT | Room temperature/ambient temperature |
| UM | Unknown morphology |
| v/v | Volume/volume |
| w/ | With |
| wt | Weight |

### B. Experimental Methods

### Example 1: Stable Form and Hydrate Screen Via Slurry-Trituration Experiments

Slurry triburation experiments target the stable forms, including stable solvates and hydrates.

The slurry-trituration experiments were conducted by stirring solids of Compound I in specified solvents and solvent mixtures at various temperatures for 7 days (elevated temperature) or 14-18 days (ambient and sub-ambient temperatures). A summary of experimental conditions and results is detailed in Table 13.

TABLE 13

| Solvent | | | XRPD |
|---|---|---|---|
| (v/v) | Condition * | Observations | Result |
| CHCl₃ | 2-8 °C, 18 d | white suspension, B/E | Form H |
| | | particles & agg., UM | |
| dioxane/ | 2-8 °C, 18 d | white suspension, B/E | Form A |
| H₂O | | particles & agg., UM | |
| (85/15) | | | |
| HFIPA/ | 2-8 °C, 18 d | white suspension, B/E | Form A |
| MTBE | | particles & agg., UM | |
| (50/50) | | | |
| TFE/H₂O | 2-8 °C, 18 d | white suspension, B/E | Form A |
| (87/13) | | particles & agg., UM | |
| THF/H₂O | 2-8 °C, 18 d | white suspension, B/E | Form A |
| (93/7) | | particles & agg., UM | |
| acetone | RT, 14 d | yellow suspension, B/E | Form A |
| | | particles & agg., UM | |
| 2-BuOH | RT, 14 d | white suspension, B/E | Form A |
| | | particles & agg., UM | |
| DCM | RT, 14 d | brown suspension, B/E | Form C, w/ |
| | | particles & agg., UM | extra peaks |
| | | | at 2.8° and |
| | | | 8.6° 2θ |
| DMSO/ | RT, 14 d | white suspension, B/E | Form A |
| iPrOAc | | particles & agg., UM | |
| (5/95) | | | |
| dioxane | RT, 14 d | white suspension, B/E | Form A |
| | | particles & agg., UM | |
| EtOH | RT, 14 d | white suspension, B/E | Form A |
| | | particles & agg., UM | |
| MEK | RT, 14 d | white suspension, B/E | Form A |
| | | particles & agg., UM | |
| MeOH | RT, 14 d | white suspension, B/E | Form A |
| | | particles & agg., UM | |
| NMP: | RT, 14 d | white suspension, B/E | Form A |
| nitromethane | | particles & agg., UM | |
| (20/80) | | | |
| 1-PrOH | RT, 14 d | white suspension, B/E | Form A |
| | | particles & agg., UM | |
| TFE | RT, 14 d | white suspension, B/E | Form A |
| | | particles & agg., UM | |
| THF | RT, 14 d | off white suspension, B/E | Form A |
| | | particles & agg., UM | |
| anh. ACN | 55 °C, 7 d | white suspension, B/E | Form A |
| | | particles & agg., UM | |
| anh. DCE | 55 °C, 7 d | light yellow suspension, | Form A |
| | | B/E particles & agg., UM | |
| anh. EtOAc | 55 °C, 7 d | white suspension, B/E | Form A |
| | | particles & agg., UM | |
| anh. EtOH | 55 °C, 7 d | brown suspension, B/E | decomposed |
| | | particles & agg., UM | |
| anh. toluene | 55 °C, 7 d | white suspension, B/E | Form A |
| | | particles & agg., UM | |

* Temperatures are approximate.

### Example 2: Polymorph Screen

Solids of Compound I unless noted otherwise, was used as the starting material. Materials produced in the study were utilized for selected experiments.

### Method a: Evaporation Experiments

Solutions of starting material were allowed to partially evaporate or evaporate to dryness at ambient or elevated temperature from open vials for fast evaporation (FE) or from vials covered with aluminum foil with pin holes for slow evaporation (SE). Prior to evaporation, solutions were filtered at ambient or elevated temperature using 0.2 μm nylon filters.

### Method b: Cooling Experiments

Solutions of starting material were prepared in specified solvents at elevated temperature using a hot plate for heating. These were typically hot-filtered through a 0.2 μm nylon filter into warm receiving vials. The vials were either quickly transferred into a sub-ambient temperature bath (typically dry ice/acetone) for crash cooling (CC), removed from the hot place for fast cooling (FC) or the heat was turned off to allow for slow cooling (SC). If solids precipitated, they were isolated cold by vacuum filtration. If the solution remained clear, the sample was either kept at sub-ambient temperatures or further crystallization techniques were applied.

### Method c: Slurry Experiments

Solids were suspended in specified solvents. The suspensions were then agitated at ambient or set temperature. After a given amount of time solids were isolated.

### Method d: Solvent/Anti-Solvent Precipitation

Solutions of starting material were prepared at ambient or elevated temperature and filtered using 0.2 μm nylon filters. They were then mixed with appropriate anti-solvents at elevated temperature. If no solids were observed, the samples were either cooled to ambient or sub-ambient temperatures or other crystallization techniques applied.

Polymorph screening was performed using various solvent-based techniques including evaporation, cooling, slurry, solvent/anti-solvent addition, and combinations of these techniques. Detailed experimental conditions, observations, and XRPD results are summarized in Table 14.

TABLE 14

| Solvent (v/v) | Conditions * | XRPD Result |
|---|---|---|
| Acetone | 1. SE then FE | Form A + Form B, w/ |
| | | an extra peak at 5.9° 2θ |
| | 1. FC (55 °C to an | Form B + minor Form |
| | ice/water bath) | A, w/extra peaks at 5.9° |
| | 2. kept at −10~−25 °C for 4 d | and 18.3° 2θ |
| | slurry, seeded w/8065-30- | Form A |
| | 03, RT for 7 d | |
| anh. Acetone | 1. CC (55 °C to −78 °C) | Form B + minor Form A |
| | 2. vial inside wall scratched | |
| | for nucleation, kept at | |
| | −10~−25 °C for 3 d | |
| acetone/H₂O | 1. SAS, API solution in | Form E + Form A |
| (68/32) | acetone into cold H₂O | |
| | 2. kept in ice/water bath for | |
| | l h | |
| | 3. kept at −10~−25 °C for l d | |
| 2-BuOH | 1. FC (55 °C to an ice/water | Form A |
| | bath) | |
| | 2. kept at −10~−25 °C for | |
| | 11 d | |
| iBuOH/ | 1. SAS, API solution in | — |
| t-BuOAc | iBuOH in ice/water bath, | |
| (2/1) | added t-BuOAc to API | |
| | solution | |
| | 2. kept at −10~−25 °C for 7 d | |
| CHCl₃ | SE | disordered |
| DCE | 1. FC (55 °C to an ice/water | Form A |
| | bath) | |
| | 2. kept at −10~−25 °C for | |
| | 11 d | |
| | 3. stirred at RT for 3 d | |
| | 4. FE (partial), stirred at | |
| | RT for 5 d | |
| | 5. FE (partial) | |
| DCM | SE | Form D + additional |
| | | phase(s) |
| | CC (RT to −78 °C) | disordered |
| | slurry, 2-8 °C for 6 d | Form H |
| DMF/MTBE | 1. SAS, API solution in | disordered, Form A + |
| (3/97) | DMF into MTBE at RT | additional phase(s) |
| | 2. kept at −10~−25 °C for | |
| | 20 d | |
| dioxane/ | 1. FC (55 °C to an ice/water | Form A |
| DMSO | bath) | |
| | 2. kept at 2-8 °C for 11 d | |
| | 1. solids in dioxane heated | Form A |
| | to 55 °C | |
| | 2. added DMSO at 55 °C | |
| | 3. FC in ice/water bath | |
| | 4. kept at 2-8 °C for 7 d | |
| dioxane/H₂O | 1. SAS, API solution in | Form A |
| (1/1) | dioxane into cold H₂O | |
| | 2. stirred at 2-8 °C for 1 d | |
| EtOAc/ | 1. solids in EtOAc heated | decomposed |
| HFIPA | to 55 °C | |
| | 2. added HFIPA (36:64), | |
| | stirred at 60 °C, 1 d | |
| | 3. FC (60 °C to an ice/water | |
| | bath) | |
| | 4. FE, w/N₂ purge | |
| EtOH | 1. FC (55 °C to an ice/water | Form B + minor Form A |
| | bath) | |
| | 2. kept at −10~−25 °C for | |
| | 11 d | |
| | 1. CC (55 °C to −78 °C) | Form B w/an extra peak |
| | 2. vial inside wall scratched | at 5.9° 2θ |
| | for nucleation, kept at | |
| | −10~−25 °C for 3 d | |
| HFIPA | SE | Form F |
| HFIPA/ | 1. SAS, API solution in | Form I |
| EtOAc, anh. | HFIPA into EtOAc | |
| (1/25) | 2. vial inside wall scratched | |
| | for nucleation, kept at | |
| | −10~−25 °C for 3 d | |
| MEK/ | 1. SAS, API solution in | Form E |
| BuOAc, anh. | MEK into BuOAc | |
| (1/1) | 2. kept at −10~−25 °C for 7 d | |
| | 3. FE, RT | |
| MEK/ | 1. SAS, API solution in | Form I w/peak shifts |
| Methylcyclo | MEK kept in ice/water | |
| hexane (2/1) | bath, added | |
| | methylcyclohexane to API | |
| | solution | |
| | 2. kept at −10~−25 °C for 3 d | |
| | 3. FE under N₂ purge for 2 h | |
| MeOH/ | 1. SAS, API solution in | Form E + Form A + |
| nitromethane | MeOH in ice/water bath, | additional phase(s) |
| (1/1) | nitromethane added to API | |
| | solution | |
| | 2. FE (partial), RT | |
| | 3. FE under N₂ purge for 1 h | |
| MeOH/ | 1. SAS, API solution in | Form E + additional |
| iBuOAc | MeOH into iBuOAc at RT | phase(s) |
| (2/1) | 2. kept at −10~−25 °C for 7 d | |
| | 3. FE, RT | |
| NMP/PrOAc | 1. API solution in NMP | — |
| (5/95) | added to PrOAc | |
| | 2. kept at −10~−25 °C for 7 d | |
| iPrOAc/ | 1. solids in iPrOAc, heated | — |
| NMP | to 55 °C | |
| | 2. added NMP at 55 °C | |
| | 3. FC in ice/water bath | |
| | 4. kept at −10~−25 °C for 7 d | |
| 1-PrOH/ | 1. solids in 1-PrOH heated | Form A |
| DMSO | to 60 °C | |
| | 2. added DMSO | |
| | 3. FC to RT | |
| | 4. kept at −10~−25 °C for | |
| | 20 d | |
| TFE | FE | Form G |
| toluene/ | 1. solids in toluene heated | — |
| DMSO | to 45 °C | |
| anh. | 2. added anh DMSO at | |
| | 45 °C | |
| | 3. FC in ice/water bath | |
| | 4. added additional anh. | |
| | toluene | |
| | 5. kept at −10~−25 °C for 7 d | |
| THF/H₂O | 1. FE | Form A |
| (93/7) | 2. air-dried on filter paper | |

* Times and temperatures are approximate.

### Example 3: Preparation of Selected Materials

Table 15 summarizes the preparation conditions for selected materials.

TABLE 15

| Solvent | Conditions * | XRPD Result |
|---|---|---|
| anh. acetone | 1. FC (55 °C to ice/water | Form E |
| | bath) | |
| | 2. kept at −10~−25 °C for 5 d | |
| | 3. FE (partial) under N₂ | |
| | purge, RT | |
| | 4. kept at −10~−25 °C for 4 h | |
| | 5. dried under vacuum at | |
| | 45 °C for 1 d | |
| DCM | 1. slurried at RT for 9 d | Form D |
| | 2. air-dried at RT for 1 h | |
| | 3. dried under vacuum at | |
| | 45 °C for 4 h | |
| EtOH | 1. FC (55 °C to ice/water | Form B, w/extra |
| | bath) | peak at 5.9° 2θ |
| | 2. kept at −10~−25 °C for 5 d | |
| | 3. solids isolated, dried | |
| | under vacuum at 45 °C for | |
| | 4 h | |

* Times and temperatures are approximate.

Table 16 summarizes the drying conditions for selected materials

TABLE 16

| Material | Condition * | XRPD Results |
|---|---|---|
| Compound I Form F | vacuum/45 °C/3 d | Compound I Form D |
| Compound I Form G | vacuum/45 °C/3 d | disordered |
| Compound I Form E + | vacuum/45 °C/1 d | Compound I Form B + |
| minor Form A | | minor Form A |
| Compound I Material G | vacuum/45 °C/1 d | disordered |
| Compound I Form C | vacuum/45 °C/1 d | Compound I Form D ± |
| | | additional peak |
| | vacuum/45 °C/1 h | Compound I Form D |
| Compound I Form I | vacuum/45 °C/1 d | Compound I Form B |
| Compound I Form I w/ | vacuum/45 °C/2 d | similar to Compound I |
| peak shifts | | Form B |

* Times and temperatures are approximate.

### Example 4: Competitive Slurry Experiment

In order to identify the thermodynamically most stable anhydrous form among Form A, Form D, Form B, and Form E, competitive slurries were performed in acetone at 2-8 °C, ambient temperature, and 45 °C.

At each condition, similar amounts of solids from the four forms/materials were slurried in pre-saturated solutions at the examined temperature conditions for 7 days; solids were then isolated and analyzed wet by XRPD. The detailed experimental conditions and XRPD results are summarized in Table 17.

TABLE 17

| Materials | Solvent * | Condition ** | XRPD Results |
|---|---|---|---|
| 9 mg Form A | acetone | 2-8 °C, 7 d | Form A |
| 6 mg Form D | | | |
| 7 mg Form B | | | |
| 6 mg Form E | | | |
| 9 mg Form A | acetone | RT, 7 d | Form A |
| 7 mg Form D | | | |
| 7 mg Form B | | | |
| 5 mg Form E | | | |
| 9 mg Form A | acetone | 45 °C, 7 d | Form A |
| 8 mg Form D | | | |
| 6 mg Form B | | | |
| 7 mg Form E | | | |

* Solutions were pre-saturated with Form A at each condition.
** Times and temperatures are approximate.

### X-Ray Powder Diffraction (XRPD)

XRPD patterns were collected with a PANalytical X'Pert PRO MPD or Empyrean diffractometer using an incident beam of Cu radiation produced using an Optix long, fine-focus source. An elliptically graded multilayer mirror was used to focus Cu Kα X-ray radiation through the specimen and onto the detector. Prior to the analysis, a silicon specimen (NIST SRM 640e) was analyzed to verify the observed position of the Si (111) peak is consistent with the NIST-certified position. A specimen of the sample was sandwiched between 3-μm-thick films and analyzed in transmission geometry. A beam-stop, short antiscatter extension, and antiscatter knife-edge were used to minimize the background generated by air. Soller slits for the incident and diffracted beams were used to minimize broadening from axial divergence. Diffraction patterns were collected using a scanning position-sensitive detector (X′Celerator) located 240 mm from the specimen and Data Collector software v. 5.5.

### Thermogravimetric Analysis and Differential Scanning Calorimetry Combination Analyses (TGA/DSC)

TGA/DSC combination analyses were performed using a Mettler Toledo TGA/DSC3+ analyzer. Temperature and enthalpy adjustments were performed using indium, tin, and zinc, and then verified with indium. Balance was verified with calcium oxalate. The sample was placed in an aluminum pan. The pan was hermetically sealed, the lid pierced, then inserted into the TG furnace. A weighed aluminum pan configured as the sample pan was placed on the reference platform. The furnace was heated under nitrogen.

### Dynamic Vapor Sorption (DVS)

Moisture sorption/desorption data were collected on a Surface Measurement System DVS Intrinsic instrument. Samples were not dried prior to analysis. For the as received lot, sorption and desorption data were collected over a range from 5% to 95% RH at 10% RH increments. The equilibrium criterion used for analysis was less than 0.0100% weight change in 5 minutes with a maximum equilibration time of 3 hours. Data were not corrected for the initial moisture content of the samples.

### Hot Stage Microscopy (HSM)

Hot stage microscopy was performed using a Linkam hot stage (FTIR 600) mounted on a Leica DM LP microscope equipped with a SPOT Insight™ color digital camera. Temperature calibrations were performed using USP melting point standards. Samples were placed on a cover glass, and a second cover glass was placed on top of the sample. As the stage was heated, each sample was visually observed using a 20× objective, 0.40 NA with crossed polarizers and a first order red compensator. Images were captured using SPOT software (v. 4.5.9).

### Polarized Light Microscopy (PLM)

PLM was performed using a Leica DM LP microscope equipped with a Spot Insight color camera. Crossed-polarized light was used with a first order red compensator. Various objectives were used to view the sample. Samples were suspended either in mineral oil or the dispersant selected for the method. Images were acquired at ambient temperature using Spot Advanced software (v.4.5.9). Micrometer bars were inserted onto the images as a reference for size. Particle sizes were measured using an eyepiece reticle scale calibrated using a NIST traceable stage micrometer.

### Proton Solution Nuclear Magnetic Resonance Spectroscopy (¹H NMR)

The solution NMR spectra were acquired with a Bruker AVANCE 600 MHz Spectrometer using DMSO-d₆.

Carbon-13 Solid State Nuclear Magnetic Resonance Spectroscopy (¹³C solid state NMR)

The ¹³C solid-state cross polarization magic angle spinning (CP/MAS) NMR spectrum was acquired at ambient temperature on an Agilent DD2-400 spectrometer (Larmor frequencies: ¹³C=100.549 MHz, ¹H=399.812 MHz). The sample was packed into a 4 mm PENCIL type zirconia rotor and rotated at 12 kHz at the magic angle. The spectrum was acquired with phase modulated (SPINAL-64) high power ¹H decoupling during the acquisition time using a ¹H pulse width of 2.6 μs (90°), a ramped amplitude cross polarization contact time of 5 ms, a 30 ms acquisition time, a 10 second delay between scans, a spectral width of 45 kHz with 2678 data points, and 1600 co-added scans. The free induction decay (FID) was processed using Agilent VnmrJ 3.2A software with 65536 points and an exponential line broadening factor of 10 Hz to improve the signal-to-noise ratio. The first three data points of the FID were back predicted using the VNMR linear prediction algorithm to produce a flat baseline. The chemical shifts of the spectral peaks were externally referenced to the carbonyl carbon resonance of glycine at 176.5 ppm.

### Infrared Spectroscopy (IR)

The IR spectrum was acquired using a Nicolet 6700 Fourier transform infrared (FT-IR) spectrophotometer (Thermo Nicolet) equipped with an Ever-Glo mid/far IR source, a potassium bromide (KBr) beamsplitter, and a deuterated triglycine sulfate (DTGS) detector. Wavelength verification was performed using NIST SRM 1921b (polystyrene). An attenuated total reflectance (ATR) accessory (Thunderdome™, Thermo Spectra-Tech) equipped with a germanium (Ge) crystal was used for data acquisition. The spectrum represents 256 co-added scans collected at a spectral resolution of 4 cm⁻¹. A background data set was acquired with a clean Ge crystal. A Log 1/R (R=reflectance) spectrum was obtained by taking a ratio of these two data sets against each other.

### XRPD Indexing

The high-resolution XRPD pattern of Compound I was indexed using X'Pert High Score Plus 2.2a (2.2.1) in this study. Indexing and structure refinement are computational studies. Agreement between the allowed peak positions, marked with red bars, and the observed peaks indicates a consistent unit cell determination. Successful indexing of the pattern indicates that the sample is composed primarily of a single crystalline phase. Space groups consistent with the assigned extinction symbol, unit cell parameters, and derived quantities are tabulated below each figure showing tentative indexing solution. To confirm the tentative indexing solution, the molecular packing motifs within the crystallographic unit cells must be determined. No attempts at molecular packing were performed.

### CONCLUSION

Multiple crystalline materials of Compound I were observed in this study, including Forms A-J.

Forms A, B, D, and E are anhydrous materials of Compound I. Among them, Form A is likely the most stable form within 2-8 °C to 45 °C based on results from competitive slurries.

Form C is likely a DCM solvate which desolvates into Form D. Form F is also a solvated material and converts to Form D upon drying.

Form I could represent a family of iso-structural solvates. It converts to Form E or solids similar to Form E upon drying.

Forms G and H are disordered crystalline materials and could be solvates. They become disordered upon drying.

While the invention has been described in connection with specific aspects thereof, it will be understood that invention is capable of further modifications and this application is intended to cover any variations, uses, or adaptations following, in general, the principles and including such departures from the present disclosure that come within known or customary practice within the art to which the invention pertains and can be applied to the essential features hereinbefore set forth, and follows in the scope of the claimed.

What is claimed is:

1. A crystalline form of {2-[3-cyclohexyl-3-(trans-4-propoxy-cyclohexyl)-ureido]-thiazol-5-ylsulfanyl}-acetic acid of Formula (I) 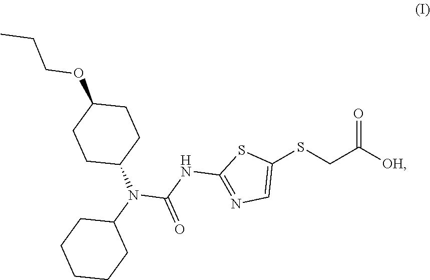 [SMILES: CCCO[C@H]1CC[C@H](N(C(=O)Nc2ncc(SCC(=O)O)s2)C2CCCCC2)CC1]
selected from the group consisting of:
a) a crystalline form characterized by an XRPD pattern having peaks at 16.9±0.2, 17.4±0.2, and 20.1±0.2 degrees two theta;
b) a crystalline form characterized by an XRPD pattern having peaks at 11.0±0.2, 11.6±0.2, and 17.8±0.2 degrees two theta;
c) a crystalline form characterized by an XRPD pattern having peaks at 4.3±0.2, 17.4±0.2, and 21.6±0.2 degrees two theta;
d) a crystalline form characterized by an XRPD pattern having peaks at 5.3±0.2, 8.7±0.2, and 26.4±0.2 degrees two theta;
e) a crystalline form characterized by an XRPD pattern having peaks at 5.8±0.2, 17.9±0.2, and 18.9±0.2 degrees two theta;
f) a crystalline form characterized by an XRPD pattern having peaks at 3.8±0.2, 9.5±0.2, and 16.8±0.2 degrees two theta;
g) a crystalline form characterized by an XRPD pattern having peaks at 3.4±0.2, 21.2±0.2, and 21.9±0.2 degrees two theta;
h) a crystalline form characterized by an XRPD pattern having peaks at 3.8±0.2, 5.3±0.2, and 8.5±0.2 degrees two theta;
i) a crystalline form characterized by an XRPD pattern having peaks at 5.0±0.2, 16.8±0.2, and 18.8±0.2 degrees two theta; and
j) a crystalline form characterized by an XRPD pattern having peaks at 5.9±0.2, 17.4±0.2, and 18.8±0.2 degrees two theta.

2. The crystalline form of claim 1, wherein the crystalline form is characterized by an XRPD pattern having peaks at 16.9±0.2, 17.4±0.2, and 20.1±0.2 degrees two theta.

3. The crystalline form of claim 2, wherein the crystalline form is characterized by an XRPD pattern having peaks at 8.7±0.2, 16.9±0.2, 17.4±0.2, and 20.1±0.2 degrees two theta.

4. The crystalline form of claim 2 or claim 3, wherein the crystalline form is characterized by an XRPD pattern substantially as shown in FIG. 1.

5. The crystalline form of any one of claims 2-4, wherein the crystalline form is characterized by an endothermic peak with onset at about 160 °C, as determined by DSC.

6. The crystalline form of any one of claims 2-5, wherein the crystalline form is characterized by a DSC profile substantially as shown in FIG. 2A.

7. The crystalline form of any one of claims 2-6, wherein the crystalline form is characterized by a TGA profile substantially as shown in FIG. 2B.

8. The crystalline form of any one of claims 2-7, wherein the crystalline form is characterized by an IR pattern having peaks at 1099.7±2.0, 1158.0±2.0, and 1313.2±2.0 cm⁻¹.

9. The crystalline form of any one of claims 2-8, wherein the crystalline form is characterized by an IR pattern having peaks at 1099.7±2.0, 1158.0±2.0, 1238.7±2.0, and 1313.2±2.0 cm⁻¹

10. The crystalline form of any one of claims 2-9, wherein the crystalline form is characterized by an IR pattern substantially as shown in FIG. 3.

11. The crystalline form of any one of claims 2-10, wherein the crystalline form is characterized by a ¹³C solid state NMR substantially as shown in FIG. 4.

12. The crystalline form of any one of claims 2-11, wherein the crystalline form is anhydrous.

13. The crystalline form of any one of claims 2-12, wherein the crystalline form has a unit cell that indexes as primitive monoclinic.

14. The crystalline form of any one of claims 2-13, wherein the crystalline form has a unit cell with an a value of about 10.193 Å, a b value of about 12.256 Å, and a c value of about 18.991 Å.

15. The crystalline form of any one of claims 2-14, wherein the crystalline form has a unit cell with a volume of about 2370.9 Å³.

16. The crystalline form of any one of claims 2-15, wherein the crystalline form is Form A.

17. The crystalline form of claim 1, wherein the crystalline form is characterized by an XRPD pattern having peaks at 11.0±0.2, 11.6±0.2, and 17.8±0.2 degrees two theta.

18. The crystalline form of claim 17, wherein the crystalline form is characterized by an XRPD pattern having peaks at 11.0±0.2, 11.6±0.2, 17.8±0.2, and 21.1±0.2 degrees two theta.

19. The crystalline form of claim 17 or claim 18, wherein the crystalline form is characterized by an XRPD pattern substantially as shown in FIG. 5.

20. The crystalline form of any one of claims 17-19, wherein the crystalline form is characterized by an endothermic peak with onset at about 166 °C, as determined by DSC.

21. The crystalline form of any one of claims 17-20, wherein the crystalline form is characterized by a DSC profile substantially as shown in FIG. 6A.

22. The crystalline form of any one of claims 17-21, wherein the crystalline form is characterized by a TGA profile substantially as shown in FIG. 6B.

23. The crystalline form of any one of claims 17-22, wherein the crystalline form is characterized by an IR pattern having peaks at 1310.1±2.0, 1514.4±2.0, and 1661.3±2.0 cm⁻¹.

24. The crystalline form of any one of claims 17-23, wherein the crystalline form is characterized by an IR pattern having peaks at 1097.3±2.0, 1310.1±2.0, 1541.4 2.0, and 1661.3±2.0 cm⁻¹

25. The crystalline form of any one of claims 17-24, wherein the crystalline form is characterized by an IR pattern substantially as shown in FIG. 7.

26. The crystalline form of any one of claims 17-25, wherein the crystalline form is characterized by a ¹³C solid state NMR substantially as shown in FIG. 8.

27. The crystalline form of any one of claims 17-26, wherein the crystalline form is anhydrous.

28. The crystalline form of any one of claims 17-27, wherein the crystalline form has a unit cell that indexes as primitive monoclinic.

29. The crystalline form of any one of claims 17-28, wherein the crystalline form has a unit cell with a value of about 11.028 Å, a b value of about 11.933 Å, and a c value of about 18.737 Å.

30. The crystalline form of any one of claims 17-29, wherein the crystalline form has a unit cell with a volume of about 2449.0 Å³.

31. The crystalline form of any one of claims 17-30, wherein the crystalline form is Form B.

32. The crystalline form of claim 1, wherein the crystalline form is characterized by an XRPD pattern having peaks at 4.3±0.2, 17.4±0.2, and 21.6±0.2 degrees two theta.

33. The crystalline form of claim 32, wherein the crystalline form is characterized by an XRPD pattern having peaks at 4.3±0.2, 8.0±0.2, 17.4±0.2, and about 21.6±0.2 degrees two theta.

34. The crystalline form of claim 32 or claim 33, wherein the crystalline form is characterized by an XRPD pattern substantially as shown in FIG. 9.

35. The crystalline form of any one of claims 32-34, wherein the crystalline form is characterized by an endothermic peak with onset at about 149 °C, as determined by DSC.

36. The crystalline form of any one of claims 32-35, wherein the crystalline form is characterized by a DSC profile substantially as shown in FIG. 10A.

37. The crystalline form of any one of claims 32-36, wherein the crystalline form is characterized by a TGA profile substantially as shown in FIG. 10B.

38. The crystalline form of any one of claims 32-37, wherein the crystalline form is a dichloromethane solvate.

39. The crystalline form of any one of claims 32-38, wherein the crystalline form has a unit cell that indexes as primitive monoclinic.

40. The crystalline form of any one of claims 32-39, wherein the crystalline form has a unit cell with a value of about 5.541 Å, a b value of about 13.040 Å, and a c value of about 40.818 Å.

41. The crystalline form of any one of claims 32-40, wherein the crystalline form has a unit cell with a volume of about 2947.6 Å³.

42. The crystalline form of any one of claims 32-41, wherein the crystalline form is Form C.

43. The crystalline form of claim 1, wherein the crystalline form is characterized by an XRPD pattern having peaks at 5.3±0.2, 8.7±0.2, and 26.4±0.2 degrees two theta.

44. The crystalline form of claim 43, wherein the crystalline form is characterized by an XRPD pattern having peaks at 5.3±0.2, 8.7±0.2, 18.2±0.2, and 26.4±0.2 degrees two theta.

45. The crystalline form of claim 43 or claim 44, characterized by an XRPD pattern substantially as shown in FIG. 11.

46. The crystalline form of any one of claims 43-45, wherein the crystalline form is characterized by an endothermic peak with onset at about 147 °C, as determined by DSC.

47. The crystalline form of any one of claims 43-46, wherein the crystalline form is characterized by a DSC profile substantially as shown in FIG. 12A.

48. The crystalline form of any one of claims 43-47, wherein the crystalline form is characterized by a TGA profile substantially as shown in FIG. 12B.

49. The crystalline form of any one of claims 43-48, wherein the crystalline form is characterized by a ¹³C solid state NMR substantially as shown in FIG. 13.

50. The crystalline form of any one of claims 43-49, wherein the crystalline form is anhydrous.

51. The crystalline form of any one of claims 43-50, wherein the crystalline form is Form D.

52. The crystalline form of claim 1, wherein the crystalline form is characterized by an XRPD pattern having peaks at 5.8±0.2, 17.9±0.2, and 18.9±0.2 degrees two theta.

53. The crystalline form of claim 52, wherein the crystalline form is characterized by an XRPD pattern having peaks at 5.8±0.2, 17.9±0.2, 18.9±0.2, and 20.7±0.2 degrees two theta.

54. The crystalline form of claim 52 or claim 53, wherein the crystalline form is characterized by an XRPD pattern substantially as shown in FIG. 14.

55. The crystalline form of any one of claims 52-54, wherein the crystalline form is characterized by an endothermic peak with onset at about 171 °C, as determined by DSC.

56. The crystalline form of any one of claims 52-55, wherein the crystalline form is characterized by a DSC profile substantially as shown in FIG. 15A.

57. The crystalline form of any one of claims 52-56, wherein the crystalline form is characterized by a TGA profile substantially as shown in FIG. 15B.

58. The crystalline form of any one of claims 52-57, wherein the crystalline form is anhydrous.

59. The crystalline form of any one of claims 52-58, wherein the crystalline form is Form E.

60. The crystalline form of claim 1, wherein the crystalline form is characterized by an XRPD pattern having peaks at 3.8±0.2, 9.5±0.2, and 16.8±0.2 degrees two theta.

61. The crystalline form of claim 60, wherein the crystalline form is characterized by an XRPD pattern having peaks at 3.8±0.2, 9.5±0.2, 16.8±0.2, and 17.9±0.2 degrees two theta.

62. The crystalline form of claim 60 or claim 61, wherein the crystalline form is characterized by an XRPD pattern substantially as shown in FIG. 16.

63. The crystalline form of any one of claims 60-62, wherein the crystalline form is a solvate.

64. The crystalline form of any one of claims 60-63, wherein the crystalline form is Form F.

65. The crystalline form of claim 1, wherein the crystalline form is characterized by an XRPD pattern having peaks at 3.4±0.2, 21.2±0.2, and 21.9±0.2 degrees two theta.

66. The crystalline form of claim 65, wherein the crystalline form is characterized by an XRPD pattern having peaks at 3.4±0.2, 21.2±0.2, 21.9±0.2, and 22.4±0.2 degrees two theta.

67. The crystalline form of claim 65 or claim 66, wherein the crystalline form is characterized by an XRPD pattern substantially as shown in FIG. 17.

68. The crystalline form of any one of claims 65-67, wherein the crystalline form is a solvate.

69. The crystalline form of any one of claims 65-68, wherein the crystalline form is Form G.

70. The crystalline form of claim 1, wherein the crystalline form is characterized by an XRPD pattern having peaks at 3.8±0.2, 5.3±0.2, and 8.5±0.2 degrees two theta.

71. The crystalline form of claim 70, wherein the crystalline form is characterized by an XRPD pattern having peaks at 3.8±0.2, 5.3±0.2, 8.5±0.2, and 15.9±0.2 degrees two theta.

72. The crystalline form of claim 70 or claim 71, wherein the crystalline form is characterized by an XRPD pattern substantially as shown in FIG. 18.

73. The crystalline form of any one of claims 70-72, wherein the crystalline form is a solvate.

74. The crystalline form of any one of claims 70-73, wherein the crystalline form is Form H.

75. The crystalline form of claim 1, wherein the crystalline form is characterized by an XRPD pattern having peaks at 5.0±0.2, 16.8±0.2, and 18.8±0.2 degrees two theta.

76. The crystalline form of claim 75, wherein the crystalline form is characterized by an XRPD pattern having peaks at 5.0±0.2, 15.9±0.2, 16.8±0.2, and 18.8±0.2 degrees two theta.

77. The crystalline form of claim 75 or claim 76, wherein the crystalline form is characterized by an XRPD pattern substantially as shown in FIG. 19.

78. The crystalline form of any one of claims 75-77, wherein the crystalline form is a solvate.

79. The crystalline form of any one of claims 75-78, wherein the crystalline form is Form I.

80. The crystalline form of claim 1, wherein the crystalline form is characterized by an XRPD pattern having peaks at 5.9±0.2, 17.4±0.2, and 18.8±0.2 degrees two theta.

81. The crystalline form of claim 80, wherein the crystalline form is characterized by an XRPD pattern having peaks at 5.9±0.2, 12.7±0.2, 17.4±0.2, and 18.8±0.2 degrees two theta.

82. The crystalline form of claim 80 or claim 81, wherein the crystalline form is characterized by an XRPD pattern substantially as shown in FIG. 20.

83. The crystalline form of any one of claims 80-82, wherein the crystalline form is characterized by an endothermic peak with onset at about 164 °C, as determined by DSC.

84. The crystalline form of any one of claims 80-83, wherein the crystalline form is characterized by a DSC profile substantially as shown in FIG. 21A.

85. The crystalline form of any one of claims 80-84, wherein the crystalline form is characterized by a TGA profile substantially as shown in FIG. 21B.

86. The crystalline form of any one of claims 80-85, wherein the crystalline form is Form J.

87. The crystalline form of any one of claims 1-86, wherein the crystalline form is substantially free of other polymorphic forms.

88. The crystalline form of any one of claims 1-86, wherein the crystalline form has a polymorphic purity of at least about 80%.

89. The crystalline form of any one of claims 1-86, wherein the crystalline form has a polymorphic purity of at least about 90%.

90. The crystalline form of any one of claims 1-86, wherein the crystalline form has a polymorphic purity of at least about 95%.

91. The crystalline form of any one of claims 1-86, wherein the crystalline form has a polymorphic purity of at least about 99%.

92. A pharmaceutical composition comprising the crystalline form of any one of claims 1-91 and a pharmaceutically acceptable carrier, diluent, or excipient, or a mixture thereof.

93. A method of treating a type of diabetes mellitus, where the method comprises administering the pharmaceutical composition of claim 92 to a patient in need thereof.

94. The method of claim 93, wherein the type of diabetes mellitus is type 1 diabetes mellitus.

95. The method of claim 93, wherein the type of diabetes mellitus is type 2 diabetes mellitus.

96. The method of any one of claims 93-95, wherein the pharmaceutical composition is administered orally.

97. The method of any one of claims 93-96, wherein the pharmaceutical composition is administered as a tablet.

98. The method of any one of claims 93-97, wherein the patient is administered up to about 2000 mg of {2-[3-cyclohexyl-3-(trans-4-propoxy-cyclohexyl)-ureido]-thiazol-5-ylsulfanyl}-acetic acid once daily.

99. The method of any one of claims 93-97, wherein the patient is administered about 100 mg to about 1500 mg of {2-[3-cyclohexyl-3-(trans-4-propoxy-cyclohexyl)-ureido]-thiazol-5-ylsulfanyl}-acetic acid once daily.

100. The method of any one of claims 93-97, wherein the patient is administered about 500 mg to about 1000 mg of {2-[3-cyclohexyl-3-(trans-4-propoxy-cyclohexyl)-ureido]-thiazol-5-ylsulfanyl}-acetic acid once daily.

101. The method of any one of claims 93-97, wherein the patient is administered about 800 mg of {2-[3-cyclohexyl-3-(trans-4-propoxy-cyclohexyl)-ureido]-thiazol-5-ylsulfanyl}-acetic acid once daily.

102. The method of any one of claims 93-97, wherein the patient is administered less than 800 mg of {2-[3-cyclohexyl-3-(trans-4-propoxy-cyclohexyl)-ureido]-thiazol-5-ylsulfanyl}-acetic acid once daily.

103. The method of any one of claims 93-97, wherein the patient is administered about 500 mg of {2-[3-cyclohexyl-3-(trans-4-propoxy-cyclohexyl)-ureido]-thiazol-5-ylsulfanyl}-acetic acid once daily.

104. The method of any one of claims 93-97, wherein the patient is administered about 300 mg of {2-[3-cyclohexyl-3-(trans-4-propoxy-cyclohexyl)-ureido]-thiazol-5-ylsulfanyl}-acetic acid once daily.

105. The method of any one of claims 93-97, wherein the patient is administered about 100 mg of {2-[3-cyclohexyl-3-(trans-4-propoxy-cyclohexyl)-ureido]-thiazol-5-ylsulfanyl}-acetic acid once daily.
